(19) United States
(12) Patent Application Publication
(10) Pub. No.: US 2008/0132549 A1
(43) Pub. Date: Jun. 5, 2008

(54) Sulphur-Linked Imidazone Compounds for the Treatment of Hiv/Aids

(75) Inventors: Lyn Howard Jones, Kent (GB); Shahid Mohammed, Kent (GB); Sandra Newman, Kent (GB); Charles Eric Mowbray, Kent (GB); Matthew Ducan Selby, Kent (GB); Paul Anthony Stupple, Kent (GB); Nigel Alan Swain, Kent (GB)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc., San Diego, CA (US)

(21) Appl. No.: 10/599,707

(22) PCT Filed: Apr. 11, 2005
(86) PCT No.: PCT/IB05/00969

§ 371 Date: Oct. 4, 2007

Related U.S. Application Data
(60) Provisional application No. 60/571,788, filed on May 17, 2004.
(60) Provisional application No. 60/645,463, filed on Jan. 19, 2005.

(30) Foreign Application Priority Data
Apr. 14, 2004 (GB) 0408303.6
Dec. 22, 2004 (GB) 0428081.4

(51) Int. Cl. A61K 31/4164; C07D 233/84; A61K 31/4439; A61P 31/18; C07D 401/04

(52) U.S. Cl. 514/341; 548/323.5; 546/274.4; 514/398

(57) ABSTRACT

This invention relates to isophthalonitrile derivatives of formula (I)

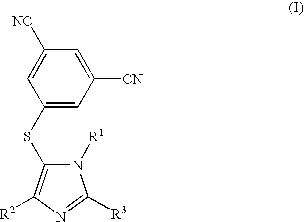

or pharmaceutically acceptable salts, solvates or derivative thereof, wherein R¹ to R³ are defined in the description, and to processes for the preparation thereof, intermediates used in their preparation of, compositions containing them and the uses of such derivatives.

The compounds of the present invention bind to the enzyme reverse transcriptase and are modulators, especially inhibitors thereof.

This invention relates to isophthalonitrile derivatives, to their use in medicine, to compositions containing them, to processes for their preparation and to intermediates used in such processes.

The compounds of the present invention bind to the enzyme reverse transcriptase and are modulators, especially inhibitors thereof. Reverse transcriptase is implicated in the infectious lifecycle of HIV, and compounds which interfere with the function of this enzyme have shown utility in the treatment of conditions including AIDS. There is a constant need to provide new and better modulators, especially inhibitors, of HIV reverse transcriptase since the virus is able to mutate, becoming resistant to the effects of known modulators.

European patent application EP 0 786 455 A1 discloses a class of imidazole compounds which inhibit the growth of HIV. Antiviral activity is ascribed to a class of N(hydroxyethyl)pyrazole derivatives in U.S. Pat. No. 3,303,200. A number of pyrazoles are disclosed as reverse transcriptase inhibitors, including: a class of N-phenylpyrazoles (J. Med. Chem., 2000, 43, 1034); a class of C and S linked aryl pyrazoles (WO02/04424); and a class of O and S linked aryl pyrazoles, the O and S aryl link being adjacent to the nitrogen atom (WO02/30907).

According to the present invention there is provided a compound of formula (I)

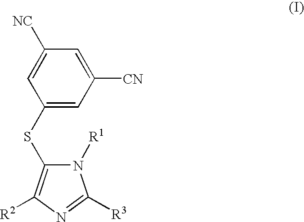

or a pharmaceutically acceptable salt, solvate or derivative thereof, wherein:
R¹ is C₁₋₄ alkyl or C₃₋₆ cycloalkyl, wherein said alkyl is optionally substituted by pyridyl or pyridyl N-oxide;
R² is C₁₋₄ alkyl, C₃₋₆ cycloalkyl, or trifluoromethyl;
R³ is —(CH₂)ₘOR⁴, —(CH₂)ₘOC(O)NH₂, —(CH₂)ₘ NH₂, or —(CH₂)ₘ NHC(O)NH₂;
R⁴ is H or C₁₋₄ alkyl;
m is 1, 2, 3 or 4.

The term “alkyl” as a group or part of a group includes straight chain and branched groups. Examples of alkyl include methyl, ethyl, n-propyl, i-propyl, n-butyl, i-butyl, sec-butyl and t-butyl. The term “C₃₋₆ cycloalkyl” means cyclopropyl, cyclobutyl, cyclopentyl, or cyclohexyl.

In one embodiment, R¹ is methyl, ethyl, i-propyl, cyclopropyl, or pyridylmethyl. In a further embodiment, R¹ is methyl, ethyl or pyridylmethyl. In yet a further embodiment, R¹ is methyl or ethyl. In yet a further embodiment, R¹ is ethyl.

In one embodiment, R² is methyl, ethyl, n-propyl, i-propyl, cyclopropyl, or trifluoromethyl. In a further embodiment, R² is ethyl, i-propyl or cyclopropyl. In yet a further embodiment, R² is ethyl. In yet a further embodiment, R² is i-propyl or cyclopropyl.

In one embodiment, R³ is —(CH₂)ₘOR⁴ or —(CH₂)ₘOC(O)NH₂. In a further embodiment, R³ is —CH₂OR⁴, —(CH₂)₂OR⁴, —CH₂OC(O)NH₂ or —(CH₂)₂OC(O)NH₂. In yet a further embodiment, R³ is —CH₂OR⁴ or —(CH₂)₂OR⁴. In yet a further embodiment, R³ is —(CH₂)₂OR⁴. In yet a further embodiment, R³ is —(CH₂)₂OC(O)NH₂.

In one embodiment, R⁴ is H. In a further embodiment, R⁴ is methyl.

It is to be understood that the invention covers all combinations of particular embodiments of the invention as described hereinabove, consistent with the definition of compounds of formula (I).

The compounds of the invention include compounds of formula (I) and pharmaceutically acceptable salts, solvates or derivatives thereof (wherein derivatives include complexes, polymorphs, prodrugs and isotopically-labeled compounds, as well as salts, solvates and salt solvates thereof), and isomers thereof. In a further embodiment, the compounds of the invention are the compounds of formula (I) and pharmaceutically acceptable salts and solvates thereof, in particular the compounds of formula (I). It is to be understood that the aforementioned compounds of the invention include polymorphs and isomers thereof.

Pharmaceutically acceptable salts of the compounds of formula (I) include the acid addition salts thereof.

Suitable acid addition salts are formed from acids which form non-toxic salts. Examples include the acetate, aspartate, benzoate, besylate, bicarbonate, bisulphate, borate, bromide, camsylate, carbonate, chloride, citrate, edisylate, esylate, formate, fumarate, gluceptate, gluconate, glucuronate, hexafluorophosphate, hibenzate, hydrobromide, hydrochloride, hydroiodide, iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, sulphate, tartrate, tosylate and trifluoroacetate salts.

Hemisalts of acids may also be formed, for example, hemisulphate salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:

(i) by reacting the compound of formula (I) with the desired acid;
(ii) by removing an acid- or base-labile protecting group from a suitable precursor of the compound of formula (I) or by ring-opening a suitable cyclic precursor, for example, a lactone or lactam, using the desired acid; or
(iii) by converting one salt of the compound of formula (I) to another by reaction with an appropriate acid or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Complexes include clathrates, i.e. drug-host inclusion complexes wherein, in contrast to the aforementioned solvates, the drug and host are present in stoichiometric or non-stoichiometric amounts. Also included are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the present invention may have the ability to crystallize in more than one form, a characteristic known as polymorphism, and all such polymorphic forms (“polymorphs”) are encompassed within the scope of the invention. Polymorphism generally can occur as a response to changes in temperature or pressure or both, and can also result from variations in the crystallization process. Polymorphs can be distinguished by various physical characteristics, and typically the X-ray diffraction patterns, solubility behavior, and melting point of the compound are used to distinguish polymorphs.

Certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of prodrugs in accordance with the invention include:

i) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
ii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types in accordance with the invention may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:

(i) where the compound of formula (I) contains a methyl group, an hydroxymethyl derivative thereof (—CH₃->—CH₂OH);
(ii) where the compound of formula (I) contains a tertiary amino group, a secondary amino derivative thereof (—NR¹R²->—NHR¹ or —NHR²);
(iii) where the compound of formula (I) contains a phenyl moiety, a phenol derivative thereof (—Ph->—PhOH); and
(iv) where the compound of formula (I) contains an amide group, a carboxylic acid derivative thereof (—CONH₂->COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all optical isomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention also includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹³F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹³F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Representative compounds of formula (I) include the compounds of examples 1, 7, 8, 11, 13, 15, 16, 17, 29, 30 and 31, and pharmaceutically acceptable salts, solvates or derivatives thereof.

In the general processes, and schemes, that follow: R¹ to R³ are as previously defined unless otherwise stated; X and Y are halo, such as chloro, bromo or iodo, OH, or any suitable leaving group; DMSO is dimethylsulphoxide; DMF is dimethylformamide; 0.88 SG=concentrated ammonium hydroxide solution, 0.88 ammonia.

Compounds of formula (I) may be prepared by any methods known for the preparation of compounds of analogous structure.

Compounds of formula (I), and intermediates thereto, may be prepared according to the schemes that follow.

It will be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

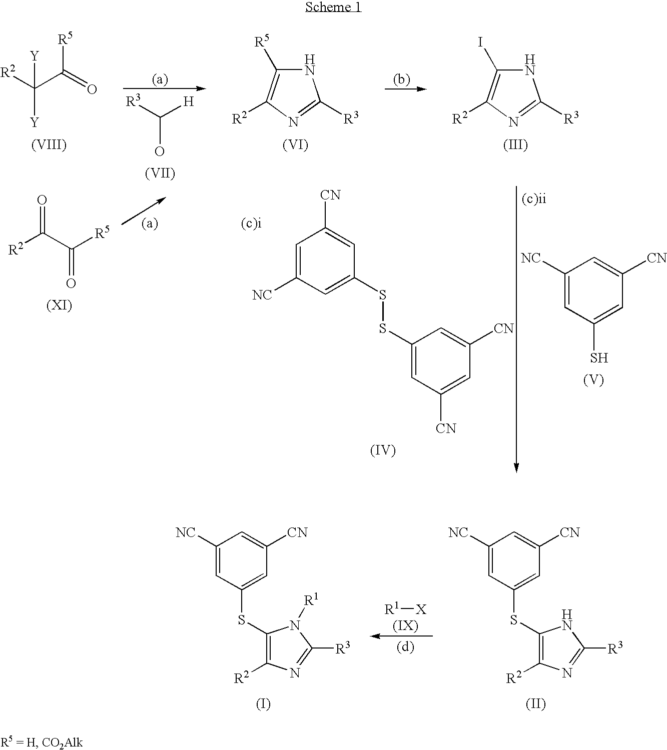

With specific reference to Scheme 1, the transformations depicted therein may be effected as follows:

### (a) Cyclisation

Compounds of formula (VI) where R⁵=H may be prepared by the reaction of a compound of formula (VII) with a compound of formula (VIII) where Y is preferably chloro, in the presence of a source of ammonia, such as concentrated ammonium hydroxide solution, 0.88 SG or ammonium acetate, under conventional conditions. The reaction is generally carried out in a suitable solvent such as acetonitrile, at ambient temperature for 18-48 h. Typical conditions comprise of 1.0 equivalent of compound (VII), 1.0 equivalent of (VIII) and excess 0.88 ammonia, in acetonitrile at room temperature for 18 h.

Compounds of formula (VI) where R⁵≠H may be prepared by the reaction of a compound of formula (VIII) where Y is OH or (XI) with a compound of formula (VII) in the presence of a source of ammonia, such as concentrated ammonium hydroxide solution, 0.88 SG or ammonium acetate, optionally in the presence of a mild base such as triethylamine, in a suitable solvent such as methanol or tetrahydrofuran, at ambient temperature for 18-48 h. Typical conditions comprise of 1.0 equivalent of compound (VII), 1.1 equivalent of compound (VIII)/(X) and excess 0.88 ammonia, in methanol, at room temperature for 18 h.

### (b) Iodination

Compounds of formula (III) may be prepared by the iodination of a compound of formula (VI) where R⁵═H using a source of iodine, such as molecular iodine (e.g. iodine), iodine with periodic acid dihydrate or N-iodosuccinimide, under conventional conditions. The reaction is optionally carried out in the presence of a suitable base such as sodium hydroxide or potassium hydroxide, in a suitable solvent such as dichloromethane, methanol or a biphasic system such chloroform and acetic acid, at a temperature between 0° C. to 60° C., for 0.5 to 4 h.

Iodination of compounds (VI) where R⁵=CO₂Alkyl is carried out by hydrolysis of the ester functionality under basic conditions and treatment of the crude carboxylic acid salt with iodine under basic conditions. Typical conditions comprise of 1.0 equivalent of compound (VI) and 10 equivalents of base such as sodium hydroxide in methanol, heated under reflux for 3 h. Concentration in vacuo followed by addition of dichloromethane and water (pH adjusted to pH12 with 2M HCl) and 1.0-1.3 equivalents of iodine, stirring at ambient temperature for 18 h.

When R²=haloalkyl, typical conditions comprise of 1.0 equivalent of compound (VI), 1.0-1.5 equivalents of iodine and 1.0 equivalent periodic acid dihydrate in a mixture of chloroform and acetic acid, heated at 60° C. for 4 h.

When R²=alkyl, typical conditions comprise of 1.0 equivalent of compound (VI), 1-1.5 equivalents of base such as sodium hydroxide and 1-1.3 equivalents of iodine in a mixture of dichloromethane and methanol, at 0° C. for 30 min.

### (c) Nucleophilic Substitution

(c)i In one embodiment of the present invention, compounds of formula (II) may be prepared by the reaction of compounds of formula (III) and the compound of formula (IV) under conventional conditions. Conveniently, the reaction may be effected using a base, such as an alkali metal base, for example, an alkali metal hydride (e.g., sodium, lithium or potassium hydride); in the presence of a solvent, such as a polar aprotic solvent (e.g., DMSO); and at ambient temperature.
(c)ii In a further embodiment, compounds of formula (II) may be prepared by the reaction of compounds of formula (III) and the compound of formula (V) under conventional conditions. Conveniently, the reaction may be effected using a base, such as an alkali metal base, for example, an alkali metal carbonate base (e.g., potassium, sodium or caesium carbonate); optionally in the presence of copper (I) iodide, in a suitable solvent such as a polar aprotic solvent (e.g., acetonitrile) at ambient temperature or elevated temperature, such as ambient temperature to reflux for 1-24 h. Typical conditions comprise of 1.0 equivalent of compound (III), 1.2-1.5 equivalents of compound (V), 1.2-1.5 equivalents of caesium carbonate, optionally in the presence of copper (I) iodide (cat.), in acetonitrile, at 25-80° C. for 1-24 h.

### (d) Alkylation

Compounds of formula (I) may be prepared by alkylating a compound of formula (II) with a compound of formula (IX) under conventional alkylating conditions. Conveniently, alkylation is effected using a base, such as an alkali metal base, for example, an alkali metal carbonate (e.g., sodium, potassium or caesium carbonate); in the presence of a solvent, such as a polar aprotic solvent (e.g., acetonitrile or DMF); and at ambient or elevated temperature, such as ambient temperature to 40° C. Typical conditions comprise of 1.0 equivalent of compound (II), 1.0-1.2 equivalents of compound (IX), 1.5-2.0 equivalents of potassium carbonate, in DMF at 25° C. for 1-24 h.

Compounds of formulae (VII) and (VIII) are either known compounds or may be prepared by conventional chemistry. The compounds of formulae (IV) and (V) may be prepared as described in the preparations section below (respectively, preparations 6 and 3).

Alternatively, compounds of formula (I) may be prepared as described in Scheme 2.

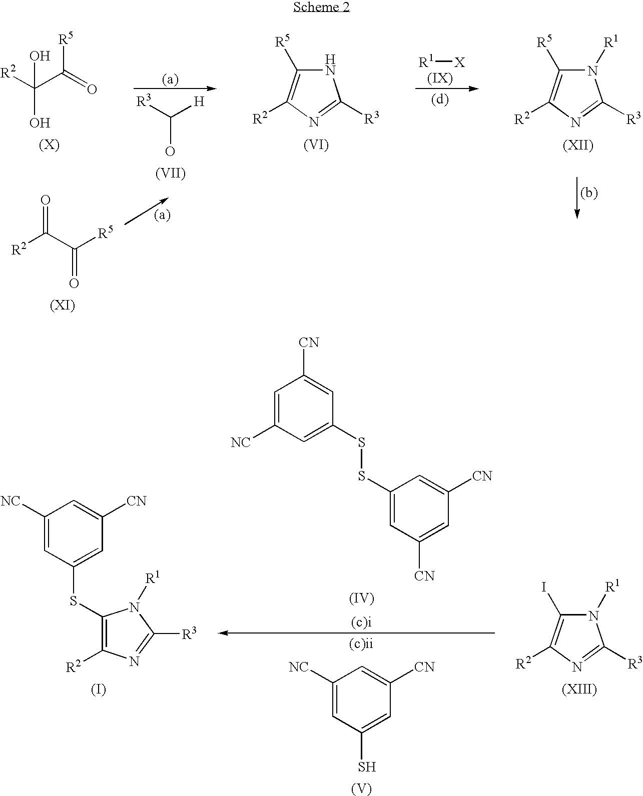

With specific reference to Scheme 2, the transformations depicted therein may be effected as follows:

### (a) Cyclisation

Compounds of formula (VI) may be prepared by the reaction of a compound of formula (X) or (XI) with a compound of formula (VII) as described in Scheme 1.

### (b) Iodination

Compounds of formula (XIII) may be prepared by the iodination of compounds of formula (XII), as described for compounds (III) in Scheme 1.

### (c) Nucleophilic Substitution

Compounds of formula (I) may be prepared by reaction of compound (XIII) with compounds of formula (IV) or (V), as described in Scheme 1.

### (d) Alkylation

Compounds of formula (XII) may be prepared by alkylation of compound (VI) with compound (IX), as described in Scheme 1.

When R²=cyclopropyl and R⁵=CO₂Alkyl, compounds of formula (X) may be prepared as described in J. Labelled Compounds and Radiopharmaceuticals 38(5), 453, 9381. Typical conditions comprise of 1.0 equivalent of suitable acid chloride, R²C(O)Cl; 1.0 equivalent of suitable ylide PPh₃=CHR⁵; and a suitable acid catalyst such as benzenesulfonic acid, in dichloromethane, under ambient conditions for 2 h. Concentration in vacuo is followed by oxidation with 1.5 equivalents Oxone® (potassium peroxymonosulphate), in a suitable biphasic system such as tetrahydrofuran:water, under ambient conditions for 18 h.

When R²=CF₃ and R⁵=H, compounds of formula (XI) may be prepared by reaction of ketone R²C(O)Z^{a}Z^{b}, preferably where Z^{a}=Z^{b}=bromo, with sodium acetate trihydrate, in a suitable solvent such as water, heated under reflux for 0.5-1 h. Typical conditions comprise of 1.0 equivalent of R²C(O)Z^{a}Z^{b} and 2.0 equivalents of sodium acetate trihydrate in water, heated under reflux for 30 min.

It will be appreciated by those skilled in the art that, as illustrated in the schemes above, it may be necessary or desirable at any stage in the synthesis of compounds of formula (I) to protect one or more sensitive groups in the molecule so as to prevent undesirable side reactions. In particular, it may be necessary or desirable to protect amino or hydroxy groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 2, pages 17-245 (“Protection for the Hydroxyl Group”), and chapter 7, pages 494-653 (“Protection for the Amino Group”), incorporated herein by reference, which also describes methods for the removal of such groups. For example, when R³ is benzyl ether, the benzyl group may be cleaved by use of 2M boron trichloride dimethylsulfide complex solution in dichloromethane, under ambient conditions. When R³ incorporates a phthalimide group, the free amine can be generated by reaction with hydrazine monohydrate, in a suitable solvent such as ethanol, at 450 for 18 h.

It will be still further appreciated that compounds of formula (I) may be converted to alternative compounds of formula (I) using standard chemical reactions and transformations. For example, when R³ is hydroxy, a series of carbamic acids are afforded by reaction with trichloroacetylisocyanate (examples 13-22). Furthermore, a series of alkoxides may also be produced by reaction with a suitable alkyl halide, in the presence of a suitable base such as sodium hydride (examples 23 and 24). Additionally, when R³ is amino, a series of ureas are afforded by reaction with trichloroacetylisocyanate (examples 43 and 44).

According to another aspect, the invention provides a process for preparing compounds of formula (I) comprising alkylation of a compound of formula (II) with a compound of formula (IX). Conveniently, alkylation is effected under the conditions described hereinabove in connection with scheme 1, step (d).

According to another aspect, the invention provides a process for preparing compounds of formula (I) comprising reaction of a compound of formula (XIII) with a compound of formula (IV) or (V). Conveniently, this reaction is effected under the conditions described hereinabove in connection with scheme 2, step (c).

The compounds of the invention are reverse transcriptase inhibitors and are therefore of use in the treatment of HIV, a retroviral infection genetically related to HIV, and AIDS.

Accordingly, in another aspect the invention provides a compound of the formula (I) or a pharmaceutically acceptable salt, solvate or derivative thereof for use as a medicament.

In another aspect the invention provides a compound of the formula (I) or a pharmaceutically acceptable salt, solvate or derivative thereof, for use as a reverse transcriptase inhibitor or modulator.

In another aspect the invention provides a compound of the formula (I) or a pharmaceutically acceptable salt, solvate or derivative thereof for use in the treatment of a HIV, a retroviral infection genetically related to HIV, or AIDS.

In another aspect the invention provides the use of a compound of the formula (I) or a pharmaceutically acceptable salt, solvate or derivative thereof in the manufacture of a medicament having reverse transcriptase inhibitory or modulating activity.

In another aspect the invention provides the use of a compound of the formula (I) or of a pharmaceutically acceptable salt, solvate or derivative thereof in the manufacture of a medicament for the treatment of a HIV, a retroviral infection genetically related to HIV, or AIDS.

In another aspect, the invention provides a method of treatment of a mammal, including a human being, with a reverse transcriptase inhibitor or modulator, which comprises treating said mammal with an effective amount of a compound of the formula (I) or a pharmaceutically acceptable salt, solvate or derivative thereof.

In another aspect the invention provides a method of treatment of a mammal, including a human being, with a HIV, a retroviral infection genetically related to HIV, or AIDS, which comprises treating said mammal with an effective amount of a compound of the formula (I) or a pharmaceutically acceptable salt, solvate or derivative thereof.

The compounds of the invention may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or in any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavours, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound comprising, for example, ethanol (optionally, aqueous ethanol) or a suitable alternative agent for dispersing, solubilising, or extending release of the compound, the propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid) (PGLA). Modified release formulations include delayed-, sustained-, pulsed-controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a compound of the invention in combination with another therapeutic agent, for example, for the purpose of treating a particular disease or condition, it is within the scope of the present invention that two or more pharmaceutical compositions, at least one of which contains a compound of the invention, may conveniently be combined in the form of a kit suitable for coadministration of the compositions.

Thus the kit of the invention comprises two or more separate pharmaceutical compositions, at least one of which contains a compound of formula (I) or a pharmaceutically acceptable salt, solvate or derivative thereof, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, having a weight of about 65 to 70 kg, the total daily dose of a compound of the invention is typically in the range 1 to 10000 mg, such as 10 to 1000 mg, for example 25 to 500 mg, depending, of course, on the mode of administration, the age, condition and weight of the patient, and will in any case be at the ultimate discretion of the physician. The total daily dose may be administered in single or divided doses.

Accordingly in another aspect the invention provides a pharmaceutical composition comprising a compound of the formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

In particular, the compounds of formula (I) are more resistant to metabolism. In providing compounds of formula (I) which exhibit increased resistance to metabolism coupled with comparable or improved potency, the invention provides compounds which are therapeutically effective NNRT is at significantly lower dosages than the compounds of the prior art. Moreover, the increased solubility of compounds of formula (I) further facilitates lower dosages and flexibility in the routes of administration. These advantages can be expected to improve efficacy, safety, and patient compliance during treatment; and reduce the cost thereof.

The compounds of formula (I) and their pharmaceutically acceptable salts, solvates and derivatives may be administered alone or as part of a combination therapy. Thus included within the scope of the present invention are embodiments comprising coadministration of, and compositions which contain, in addition to a compound of the invention, one or more additional therapeutic agents. Such multiple drug regimens, often referred to as combination therapy, may be used in the treatment and prevention of infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of the invention are coadministered in combination with one or more additional therapeutic agents such as those described in detail further herein.

In a further embodiment of the invention, combinations of the present invention include treatment with a compound of formula (I), or a pharmaceutically acceptable salt, solvate or derivative thereof, and one or more additional therapeutic agents selected from the following: HIV protease inhibitors (PIs), including but not limited to indinavir, ritonavir, saquinavir, nelfinavir, lopinavir, amprenavir, atazanavir, tipranavir, AG1859 and TMC 114; non-nucleoside reverse transcriptase inhibitors (NNRTIs), including but not limited to nevirapine, delavirdine, capravirine, efavirenz, GW-8248, GW-5634 and etravirine; nucleoside/nucleotide reverse transcriptase inhibitors, including but not limited to zidovudine, didanosine, zalcitabine, stavudine, lamivudine, abacavir, adefovir dipivoxil, tenofovir and emtricitabine; CCR5 antagonists, including but not limited to:
N-{(1S)-3-[3-(3-isopropyl-5-methyl-4H-1,2,4-triazol-4-yl)-exo-8-azabicyclo[3.2.1]oct-8-yl]-1-phenylpropyl}-4,4-difluorocyclohexanecarboxamide or a pharmaceutically acceptable salt, solvate or derivative thereof,
methyl 1-endo-{8-[(3S)-3-(acetylamino)-3-(3-fluorophenyl)propyl]-8-azabicyclo[3.2.1]oct-3-yl}-2-methyl-1,4,6,7-tetrahydro-5H-imidazo[4,5-c]pyridine-5-carboxylate or a pharmaceutically acceptable salt, solvate or derivative thereof,
ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or a pharmaceutically acceptable salt, solvate or derivative thereof,
   Sch-D, ONO-4128, AMD-887, GW-873140 and CMPD-167; CXCR4 antagonists, including but not limited to AMD-3100, AMD-070, and KRK-2731; integrase inhibitors, including but not limited to L-870,810; entry (e.g. fusion) inhibitors, including but not limited to enfuviritide; agents which inhibit the interaction of gp120 and CD4, including but not limited to BMS806 and BMS-488043; and RNaseH inhibitors.

There is also included within the scope the present invention, combinations of a compound of formula (I), or a pharmaceutically acceptable salt, solvate or derivative thereof, together with one or more additional therapeutic agents independently selected from the group consisting of proliferation inhibitors, e.g. hydroxyurea; immunomodulators, such as granulocyte macrophage colony stimulating growth factors (e.g. sargramostim), and various forms of interferon or interferon derivatives; other chemokine receptor agonists/antagonists such as CXCR4 antagonists, e.g. AMD-3100, AMD-070 or KRK-2731; tachykinin receptor modulators (e.g. NK1 antagonists) and various forms of interferon or interferon derivatives; inhibitors of viral transcription and RNA replication; agents which influence, in particular down regulate, CCR5 receptor expression; chemokines that induce CCR5 receptor internalisation such MIP-1α, MIP-1β, RANTES and derivatives thereof; and other agents that inhibit viral infection or improve the condition or outcome of HIV-infected individuals through different mechanisms.

Agents which influence (in particular down regulate) CCR5 receptor expression include immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab); and agents which interfere with cell activation or cell cycling, such as rapamycin.

There is also included within the scope the present invention, combinations of a compound of formula (I), or a pharmaceutically acceptable salt, solvate or derivative thereof, together with one or more additional therapeutic agents which yet further slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir, saquinavir or ketoconazole.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more other NNRTIs; one or more NRTIs and a PI; one or more NRTIs and a CCR5 antagonist; a PI; a PI and an NNRTI; and so on.

In addition to the requirement of therapeutic efficacy, which may necessitate the use of therapeutic agents in addition to the compounds of the invention, there may be additional rationales which compel or highly recommend the use of a combination of a compound of the invention and another therapeutic agent, such as in the treatment of diseases or conditions which directly result from or indirectly accompany the basic or underlying disease or condition. For example, it may be necessary or at least desirable to treat Hepatitis C Virus (HCV), Hepatitis B Virus (HBV), Human Papillomavirus (HPV), opportunistic infections (including bacterial and fungal infections), neoplasms, and other conditions which occur as the result of the immune-compromised state of the patient being treated. Other therapeutic agents may be used with the compounds of the invention, e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include: interferons, pegylated interferons (e.g. peginterferon alfa-2a and peginterferon alfa-2b), lamivudine, ribavirin, and emtricitabine for the treatment of hepatitis; antifungals such as fluconazole, itraconazole, and voriconazole; antibacterials such as azithromycin and clarithromycin; interferons, daunorubicin, doxorubicin, and paclitaxel for the treatment of AIDS related Kaposi's sarcoma; and cidofovir, fomivirsen, foscarnet, ganciclovir and valcyte for the treatment of cytomegalovirus (CMV) retinitis.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab); a HMG CoA reductase inhibitor, such as a statin (e.g. atorvastatin); or an immunosuppressant, such as cyclosporin or a macrolide such as tacrolimus.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

It is to be appreciated that all references herein to treatment include curative, palliative and prophylactic treatment.

The invention is illustrated by the following Examples and Preparations in which the following further abbreviations may be used:
h=hour
min=minute
rt=room temperature
LRMS=low resolution mass spectrum
HLRMS=high resolution mass spectrum
APCI+=atmospheric pressure chemical ionisation
ESI+=electrospray ionisation
LCMS=liquid chromatography-mass spectroscopy
TLC=thin layer chromatography
Me=methyl
Liquid chromatography-mass spectroscopy (LCMS) conditions were:
Column: Phenomenex Luna 3 u C18 100 A, 50×2.00 mm 3 u Micron;
Eluent: 5 min gradient method, Solvent A: 0.1% Formic Acid+Water, Solvent B: 0.1% Formic Acid+acetonitrile, 1 ml/min.

### EXAMPLE 1

### 5-[3,5-Diethyl-2-(2-hydroxyethyl)-3H-imidazol-4-ylsulfanyl]-isophthalonitrile

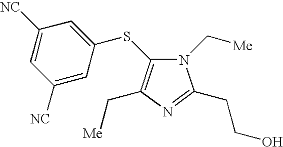

Ferric chloride (214 mg, 1.3 mmol) was added in one portion to a solution of the compound of Preparation 8 (110 mg, 0.26 mmol) in dichloromethane (4 mL), and the reaction stirred at rt for 10 min. After this time further ferric chloride (214 mg, 1.3 mmol) was added in one portion and the resulting mixture stirred at rt for 5 min. The mixture was diluted with ethyl acetate (20 mL) and washed with saturated sodium bicarbonate solution (20 mL). The layers were separated and the aqueous layer was washed with ethyl acetate (2×20 mL). The combined organic solution was washed with water (10 mL) then saturated brine (10 mL), dried over magnesium sulphate, filtered and evaporated under reduced pressure. The residue was purified by chromatography on silica gel using dichloromethane:methanol:0.88 ammonia (93:7:0.7). This afforded impure title compound as a yellow solid which was subjected to a second round of silica gel chromatography using an elution gradient of n-pentane:ethyl acetate (1:1 to 1:2), then an elution gradient of methanol:ethyl acetate (5:95) to afford the title compound as a colourless foam (32 mg).

LRMS: m/z APCI-327 [M+H]⁺.

### EXAMPLES 2 AND 3

The following compounds were prepared according to the method described above for Example 1 using the compound of Preparations 9 and 10 respectively:

### EXAMPLE 2

### 5-[5-Ethyl-2-(2-hydroxyethyl)-3-(pyridin-2-ylmethyl)-3H-imidazol-4-ylsulfanyl]-isophthalonitrile

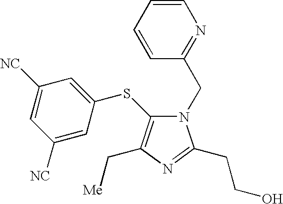

LCMS: retention time=2.21 min. m/z ESI 390 [M+H]⁺.

### EXAMPLE 3

### 5-[5-Ethyl-2-(2-hydroxyethyl)-3-methyl-3H-imidazol-4-ylsulfanyl]-isophthalonitrile

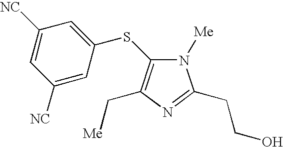

LCMS: retention time=1.95 min. m/z ESI 313 [M+H]⁺.

### EXAMPLE 4

### Carbamic acid 2-[5-(3,5-dicyano-phenylsulfanyl)-4-ethyl-1-methyl-1H-imidazol-2-yl]-ethyl ester

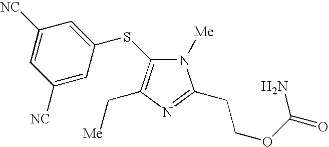

A solution of the compound of Example 3 (200 mg 0.67 mmol) was dissolved in dichloromethane (6 mL) and treated, at 0° C., with trichloroacetylisocyanate (79 μL, 0.67 mmol). After stirring this mixture for 2 h at 0° C., trichloroacetylisocyanate (79 μL, 0.67 mmol) was added and the reaction mixture stirred for 10 min before pouring onto a pad of alumina (Brockmann I, neutral alumina treated with 3% w/w water and stirred for 4 days). After 10 min ethyl acetate (70 mL) was passed through the pad of alumina and the filtrate evaporated to dryness to give a clear oil (223 mg) that solidified upon standing. This residue was partitioned between ethyl acetate (50 mL) and Na₂CO₃ (10% w/v aqueous solution, 50 mL) and the layers separated. The aqueous layer was extracted with ethyl acetate (50 mL) and the combined organic layers were washed with brine, dried over Na₂SO₄, filtered and evaporated under reduced pressure. The residue was recrystallised from ethyl acetate (20 mL) to yield the title compound (89 mg) as a white solid.

LCMS: retention time=2.37 min. m/z ESI 356 [M+H]⁺.

### EXAMPLE 5

### 5-[3,5-Diethyl-2-(2-hydroxyethyl)-3H-imidazol-4-ylsulfanyl]-isophthalonitrile

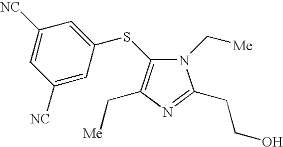

To a solution of the compound of Preparation 8 (65 g 156 mmol) in dichloromethane (800 mL) was added a solution of boron trichloride-methylsulphide complex in dichloromethane (156 ml of a 2M solution, 312 mmol), dropwise, with stirring, over 25 min. The resulting solution was stirred at rt for 3 h and then poured into saturated aqueous sodium bicarbonate solution (1 L). The layers were separated and the aqueous layer was further extracted with dichloromethane (3×200 mL). The combined organic fraction was washed with saturated brine (800 mL) and dried over magnesium sulphate, filtered and evaporated under reduced pressure. The residue was purified by chromatography on silica gel using an elution gradient of methanol:ethyl acetate (0:1 to 1:10) to afford a solid. This solid was recrystallised from a mixture of ethyl acetate and pentane to afford the title compound as a white solid (28 g). The

LRMS data was identical to that for the title compound of Example 1.

### EXAMPLE 6

### 5-(3,5-Diethyl-2-hydroxymethyl-3H-imidazol-4-ylsulfanyl)-isophthalonitrile

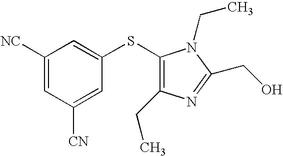

Boron trichloride-methyl sulphide complex solution (2M in dichloromethane, 2.5 mL, 5 mmol) was added to a solution of the compound of Preparation 19 (500 mg, 1.24 mmol) in dichloromethane (12 mL) and the mixture was stirred for 18 h at rt. The reaction was then quenched with sodium hydrogen carbonate solution (40 mL) and the mixture was stirred for a further 2 h. The layers were separated and the aqueous layer was extracted with dichloromethane (2×20 mL). The combined organic solution was washed with brine (20 mL), dried over magnesium sulphate and concentrated in vacuo. The residue was purified by column chromatography on silica gel, eluting with pentane:ethyl acetate, 50:50 to 0:100 followed by dichloromethane:methanol 95:5. The relevant fractions were evaporated under reduced pressure and the residue was re-crystallised from ethanol/water and dried in vacuo at 100° C. to afford the title compound as a solid in 54% yield (210 mg).

LRMS: m/z APCI-313 [M+H]⁺ Melting point; 141-143° C.

### EXAMPLE 7

### 5-[5-Cyclopropyl-3-ethyl-2-(2-hydroxy-ethyl)-3H-imidazol-4-ylsulfanyl]-isophthalonitrile

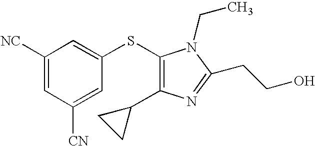

Boron trichloride-methyl sulphide complex solution (2M in dichloromethane, 2.5 mL, 5 mmol) was added to a solution of the compound of Preparation 38 (655 mg, 1.53 mmol) in dichloromethane (15 mL) and the mixture was stirred for 4.5 h at rt. The reaction was then quenched by the addition of sodium hydrogen carbonate solution (20 mL) and the layers were separated. The aqueous layer was extracted with dichloromethane (15 mL) and the combined organic solution was washed with brine (10 mL), dried over magnesium sulphate and concentrated in vacuo. The residue was purified by column chromatography on silica gel, eluting with ethyl acetate:dichloromethane:methanol, 100:0:0 to 0:98:2 to 0:95:5. The relevant fractions were evaporated under reduced pressure and the residue was triturated in a mixture of pentane/diethyl ether, filtered and dried in vacuo at 50° C. to afford the title compound as a solid in 80% yield (410 mg).

LRMS: m/z APCI-339 [M+H]⁺. Melting point; 122-124° C.

### EXAMPLE 8

### 5-(3-Ethyl-2-hydroxymethyl-5-isopropyl-3H-imidazol-4-ylsulfanyl)-isophthalonitrile

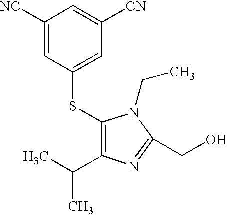

Boron trichloride-methyl sulphide complex solution (2M in dichloromethane, 1.75 mL, 3.5 mmol) was added to a solution of the compound of Preparation 49 (0.73 g, 1.75 mmol) in dichloromethane (15 mL) and the mixture was stirred for 2 h at rt. Additional boron trichloride-methyl sulphide complex solution (2M in dichloromethane, 1 mL, 2 mmol) was then added to the mixture and stirring continued for a further 4 h. The reaction was then quenched with sodium hydrogen carbonate solution (40 mL) and the mixture was stirred for 15 min. The layers were separated and the aqueous layer was extracted with dichloromethane (50 mL). The combined organic solution was washed with brine (20 mL), dried over magnesium sulphate and concentrated in vacuo. The residue was purified by column chromatography on silica gel, eluting with pentane:ethyl acetate, 50:50:0 to 0:100:0, then eluting with dichloromethane:methanol 95:5. The relevant fractions were evaporated under reduced pressure and the residue was triturated with a mixture of pentane/diethyl ether, 75:25, and dried in vacuo at 45° C. to afford the title compound as a solid in 42% yield (242 mg).

LRMS: m/z APCI-327 [M+H]⁺. Melting point; 167-169° C.

### EXAMPLES 9 TO 12

The following compounds of the general formula shown below were prepared by a method similar to that described for example 6 using the appropriate starting material and boron trichloride-methyl sulphide complex solution. The reactions were monitored by TLC analysis and were stirred at room temperature for 3-18 h.

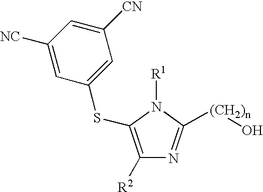

| | | | | Data and preparation | |
|---|---|---|---|---|---|
| | | | | number of | |
| No. | R¹ | R² | n | starting material. | Yield |
| 9 | CH₂CH₃ | CF₃ | 1 | LRMS: m/z APCl 353 | 75% |
| | | | | [M + H]⁺ | |
| | | | | Melting point 184-186° C. | |
| | | | | Preparation 20. | |
| 10 | CH₃ | CH(CH₃)₂ | 1 | LRMS: m/z APCl 313 | 90% |
| | | | | [M + H]⁺ | |
| | | | | Melting point 198-200° C. | |
| | | | | Preparation 50. | |
| 11 | CH₂CH₃ | CF₃ | 2 | LRMS: m/z APCl 367 | 43% |
| | | | | [M + H]⁺ | |
| | | | | Melting point 155-157° C. | |
| | | | | Preparation 57. | |
| 12 | CH₃ | CF₃ | 2 | HRMS: m/z ESI 353.0678 | 33% |
| | | | | [M + H]⁺ | |
| | | | | Preparation 58. | |

### EXAMPLE 13

### Carbamic acid 4-cyclopropyl-5-(3,5-dicyano-phenylsulfanyl)-1-ethyl-1H-imidazol-2-ylmethyl ester

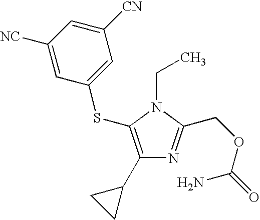

An ice-cold solution of the compound of Example 46 (100 mg 0.31 mmol) in tetrahydrofuran (2 mL) was treated with trichloroacetylisocyanate (44 μL, 0.37 mmol) and the mixture was stirred for 3 h. After this time, further trichloroacetylisocyanate (22 μL, 0.19 mmol) was added and stirring continued for 1 h. The reaction mixture was then diluted with dichloromethane (10 mL) and quenched with saturated sodium hydrogen carbonate solution (10 mL). The aqueous layer was separated and extracted with dichloromethane (10 mL) and the combined organic solution was poured onto a pad of alumina (Brockmann I, neutral alumina treated with 3% w/w water and stirred for 4 days). After 10 min ethyl acetate:methanol, 100:0 to 90:10, were passed through the pad of alumina and the combined filtrate was concentrated in vacuo. The residue was purified by column chromatography on silica gel, eluting with ethyl acetate:pentane, 50:50, followed by dichloromethane:methanol, 95:5, to give a colourless glass. The glass was then triturated in pentane:diethyl ether, 75:25, with a few drops of dichloromethane to afford the title compound as a white powder in 40% yield (45 mg).

LRMS: m/z APCI-368 [M+H]⁺. Melting point 170-172° C.

### EXAMPLE 14

### Carbamic acid 4-cyclopropyl-5-(3,5-dicyano-phenylsulfanyl)-1-methyl-1H-imidazol-2-ylmethyl ester

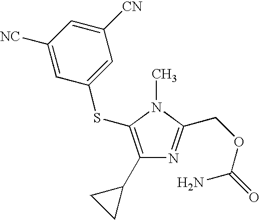

The title compound was prepared from the compound of Example 32 and trichloroacetylisocyanate, using a similar method to that of Example 13, as a solid in 65% yield.

LRMS: m/z APCI-354 [M+H]⁺

### EXAMPLE 15

### Carbamic acid 5-(3,5-dicyano-phenylsulfanyl)-1-ethyl-4-isopropyl-1H-imidazol-2-ylmethyl ester

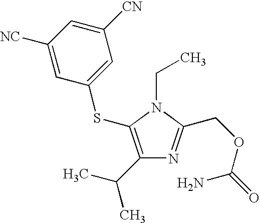

A solution of the compound of Example 8 (500 mg, 1.53 mmol) in tetrahydrofuran (15 mL) was cooled to −10° C. and treated with trichloroacetylisocyanate (215 μL, 1.84 mmol). The mixture was allowed to warm to rt and was stirred for 2 h. The reaction was then quenched with water and diluted with dichloromethane. The aqueous layer was separated and extracted with dichloromethane and the combined organic solution was poured onto a pad of alumina (Brockmann I, neutral alumina treated with 3% w/w water and stirred for 4 days). After 10 min, ethyl acetate:methanol, 100:0 to 90:10, were passed through the pad of alumina and the combined filtrate was concentrated in vacuo to give a white solid. The solid was then triturated in pentane:ethyl acetate, 75:25, to afford the title compound as a white solid in 80% yield (450 mg).

LRMS: m/z APCI-370 [M+H]⁺. Melting point 174-175° C.

### EXAMPLES 16 TO 22

The following compounds of the general formula shown below were prepared by a method similar to that described for example 4 using the appropriate alcohol and trichloroacetylisocyanate

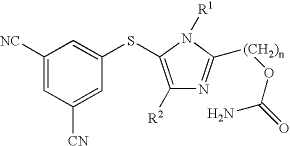

| | | | | Data and | |
|---|---|---|---|---|---|
| | | | | example number | |
| No. | R¹ | R² | n | of starting material. | Yield |
| 16 | CH₂CH₃ | CH₂CH₃ | 1 | LRMS: m/z APCl 356 | 40% |
| | | | | [M + H]⁺ | |
| | | | | Example 6. | |
| 17 | CH₂CH₃ | CF₃ | 1 | LRMS: m/z APCl 396 | 81% |
| | | | | [M + H]⁺ | |
| | | | | Example 9. | |
| 18 | CH₂CH₃ | 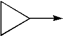 | 2 | LRMS: m/z APCl 382[M + H]⁺Example 7. | 43% |
| 19 | CH₃ | (CH₃)₂CH | 1 | LRMS: m/z APCl 356 | 70% |
| | | | | [M + H]⁺ | |
| | | | | Example 10. | |
| 20 | 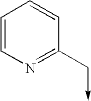 | (CH₃)₂CH | 1 | LRMS: m/z APCl 433[M + H]⁺Example 28. | 98% |
| 21 | 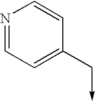 | (CH₃)₂CH | 1 | LRMS: m/z APCl 433[M + H]⁺Example 29. | 17% |
| 22 | CH₃ | (CH₃)₂CH | 2 | LRMS: m/z ES 370 | 21% |
| | | | | [M + H]⁺ | |
| | | | | Example 30. | |

### EXAMPLE 23

### 5-[3,5-Diethyl-2-(2-methoxy-ethyl)-3H-imidazol-4-ylsulfanyl]-isophthalonitrile

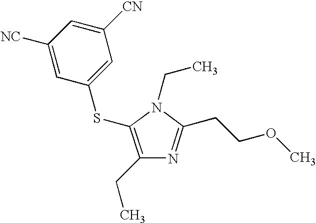

Sodium hydride (60% dispersion in mineral oil, 20 mg, 0.50 mmol) and methyl iodide (48 μL, 0.76 mmol) were added to an ice-cold solution of the compound of Example 5 (150 mg, 0.46 mmol) in N,N-dimethylformamide (1.5 mL) and the mixture was stirred at rt for 18 h. The reaction mixture was then diluted with water (25 mL) and extracted with ethyl acetate (3×25 mL). The combined organic solution was washed with water (25 mL) and brine (25 mL), dried over magnesium sulphate and concentrated in vacuo. Purification of the residue by column chromatography on silica gel, eluting with dichloromethane:methanol:0.88 ammonia, 100:0:0 to 95:5:0.5, afforded the title compound in 58% yield (90 mg) as a colourless gum.

LRMS: m/z APCI-341 [M+H]⁺

### EXAMPLE 24

### 5-(3-Ethyl-5-isopropyl-2-methoxymethyl-3H-imidazol-4-visulfanyl)-isophthalonitrile

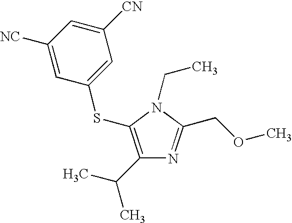

The title compound was prepared from the compound of Example 8, using a similar method to that of Example 23. The crude compound was purified by column chromatography on silica gel, eluting with pentane:ethyl acetate, 50:50, followed by trituration in diethyl ether:pentane, 75:25, to afford the desired product as a white solid in 60% yield.

LRMS: m/z APCI-341 [M+H]⁺. Melting point 156-158° C.

### EXAMPLE 25

### 5-(2-Aminomethyl-3,5-diethyl-3H-imidazol-4-ylsulfanyl)-isophthalonitrile

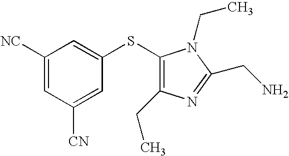

Molecular sieves (4 A, 100 mg) and hydrazine monohydrate (118 μL, 2.4 mmol) were added to a suspension of the compound of Preparation 21 (211 mg, 0.48 mmol) in ethanol (4 mL) and the mixture was heated at 45° C. for 18 h. The reaction mixture was then filtered and the residue washed with ethyl acetate (20 mL). The filtrate was diluted with further ethyl acetate (10 mL), washed with water (40 mL) and the aqueous layer was re-extracted with ethyl acetate (20 mL×2). The combined organic solution was washed with brine (10 mL) and purified directly by column chromatography on silica gel, eluting with dichloromethane:methanol:0.88 ammonia, 95:5:0.5 to 90:10:1 to afford the title compound as a white solid in 33% yield (50 mg).

LRMS: m/z APCI-312 [M+H]⁺. Melting point 133-135° C.

### EXAMPLE 26

### 5-(5-Ethyl-2-hydroxymethyl-3-(pyridin-2-ylmethyl)-3H-imidazol-4-ylsulfanyl)-isophthalonitrile

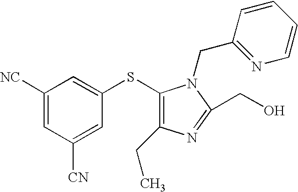

Potassium carbonate (580 mg, 4.2 mmol) was added to a solution of the compound of Preparation 23 (400 mg, 1.41 mmol) and 2-(chloromethyl)pyridine hydrochloride (254 mg, 1.55 mmol) in N,N-dimethylformamide (3 mL) and the suspension was stirred for 20 h at rt. The reaction mixture was then diluted with water and extracted into ethyl acetate (50 mL, 2×20 mL). The combined organic solution was washed with water and brine, dried over magnesium sulphate and concentrated in vacuo. The residue was purified by column chromatography on silica gel, eluting with dichloromethane:methanol: 0.88 ammonia, 95:5:0.5, followed by trituration in a mixture of diisopropyl ether and ethyl acetate, to afford the title compound in 10% yield (55 mg).

LRMS: m/z APCI-376 [M+H]⁺

### EXAMPLE 27

### Carbamic acid 5-(3,5-dicyano-phenylsulfanyl)-4-ethyl-1-(pyridin-2-ylmethyl)-1H-imidazol-2-ylmethyl ester

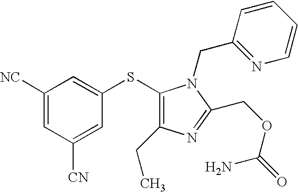

The title compound was prepared from the compound of Example 26 and trichloroacetylisocyanate, using a method similar to that of example 4, as a solid in 27% yield.

LRMS: m/z APCI-419 [M+H]⁺

### EXAMPLES 28 TO 40

The following compounds of the general formula shown below were prepared by a method similar to that described for Example 26, using a suitable imidazole and a suitable alkyl halide.

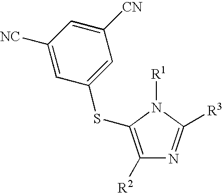

| | | | | Data (starting material | |
|---|---|---|---|---|---|
| | | | | and modifications to | |
| No. | R¹ | R² | R³ | method) | Yield |
| 28 | 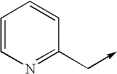 | (CH₃)₂CH | CH₂OH | LRMS: m/z APCl 390[M + H]⁺Preparation 24. | 55% |
| 29 | 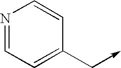 | (CH₃)₂CH | CH₂OH | LRMS: m/z APCl 390[M + H]⁺Preparation 24. | 34% |
| 30 | CH₃ | (CH₃)₂CH | CH₂CH₂OH | LRMS: m/z ES 327 | 42% |
| | | | | [M + H]⁺ | |
| | | | | Preparation 48. | |
| | | | | (a) | |
| 31 | CH₂CH₃ | (CH₃)₂CH | CH₂CH₂OH | LRMS: m/z ES 341 | 38% |
| | | | | [M + H]⁺ | |
| | | | | Preparation 48. | |
| 32 | CH₃ | 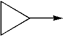 | CH₂OH | LRMS: m/z APCl 311[M + H]⁺Preparation 25. | 32% |
| 33 | CH₃ | 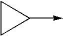 | CH₂CH₂OH | LRMS: m/z APCl 325[M + H]⁺Preparation 39. | 67% |
| 34 | 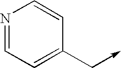 | CH₂CH₃ | CH₂CH₂OH | LRMS: m/z APCl 390[M + H]⁺Preparation 40. | 51% |
| 35 | CH₂CH₃ | 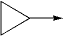 | CH₃ | LRMS: m/z APCl 309[M + H]⁺Preparation 35. | 41% |
| 36 | CH₂CH₃ | CF₃ | CH₃ | LRMS: m/z APCl 337 | 51% |
| | | | | [M + H]⁺ | |
| | | | | Preparation 55. | |
| 37 | CH₃ | 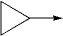 | CH₃ | LRMS: m/z APCl 295[M + H]⁺Preparation 35.(b) | 72% |
| 38 | CH₃ | CF₃ | CH₃ | LRMS: m/z APCl 323 | 51% |
| | | | | [M + H]⁺ | |
| | | | | Preparation 55. | |
| 39 | 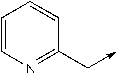 | 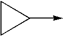 | CH₃ | LRMS: m/z APCl 372[M + H]⁺Preparation 35.(c) | 45% |
| 40 | 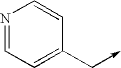 | 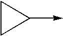 | CH₃ | LRMS: m/z APCl 372[M + H]⁺Preparation 35.(d) | 14% |

(a) Purified by column chromatography on silica gel, eluting with toluene: ethyl acetate, 100:0 to 0:100.
(b) Further methyl iodide (0.5 eq) was added after 18 h and stirring continued for a total of 36 h.
(c) Further potassium carbonate (1 eq) and 2-(bromomethyl)pyridine hydrogen bromide (0.2 eq) were added after 18 h; further 2-(bromomethyl)pyridine hydrogen bromide (0.2 eq) was added after 42 h and stirring continued for a total of 66 h.
(d) Further potassium carbonate (1 eq) and 4-(chloromethyl)pyridine hydrogen chloride (0.2 eq) were added after 18 h, further 4-(chloromethyl)pyridine hydrogen chloride (0.2 eq) was added after 42 h and stirring continued for a total of 66 h.

### EXAMPLE 41

### 5-(2-Aminomethyl-5-cyclopropyl-3-ethyl-3H-imidazol-4-visulfanyl)-isophthalonitrile

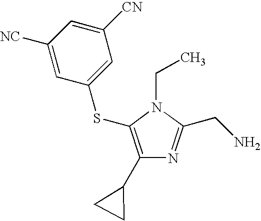

A solution of diisopropyl azodicarboxylate (150 μL, 0.72 mmol) in tetrahydrofuran (1 mL) was added to a mixture of the compound of Example 46 (144 mg, 0.77 mmol), polymer supported triphenylphosphine (260 mg, 0.77 mmol) and phthalimide (114 mg, 0.77 mmol) in tetrahydrofuran (7 mL) and the mixture was stirred at rt for 4 h. The reaction mixture was then filtered, washing through with dichloromethane (50 mL) and the filtrate was concentrated in vacuo. Purification of the residue by column chromatography on silica gel, eluting with dichloromethane:methanol, 100:0 to 98:2, afforded a white solid intermediate. Hydrazine monohydrate (75 mg, 1.5 mmol) was added to a solution of the intermediate in ethanol (5 mL) and the mixture was stirred at 45° C. for 1 h. TLC analysis indicated that starting material remained and so further hydrazine monohydrate was added (75 mg, 1.5 mmol) and the resulting solution was heated at 55° C. for 2 h. The mixture was then cooled to rt and filtered, and the solid residue was washed through with ethanol. The filtrate was concentrated in vacuo and the residue was purified by column chromatography on silica gel, eluting with ethyl acetate:dichloromethane:methanol, 100:0:0 to 0:98:2 to 0:90:10, to afford the title compound as a yellow foam in 63% yield (60 mg).

LRMS: m/z APCI-324 [M+H]⁺

### EXAMPLE 42

### 5-(2-Aminomethyl-5-cyclopropyl-3-ethyl-3H-imidazol-4-ylsulfanyl)-isophthalonitrile tartrate

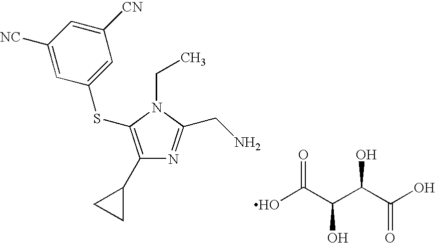

L-Tartaric acid (28 mg, 0.19 mmol) was added to a solution of the compound of Example 41 (60 mg, 0.19 mmol) in acetone (2 mL). The suspension was heated until a clear solution was formed and was then left to cool to rt. The resulting yellow precipitate was collected by filtration and washed with ice-cold acetone to afford the title compound as a solid in 42% yield (36 mg).

LRMS: m/z APCI-324 [M+H]⁺. Melting point: 190-192° C.

### EXAMPLE 43

### [4-Cyclopropyl-5-(3,5-dicyano-phenylsulfanyl)-1-ethyl-1H-imidazol-2-ylmethyl]-urea

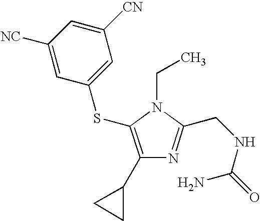

The title compound was prepared from the compound of Example 41 and trichloroacetylisocyanate, using a similar method to that of Example 4 in 0.4% yield.

LRMS: m/z APCI-367 [M+H]⁺.

### EXAMPLE 44

### [5-(3,5-Dicyano-phenylsulfanyl)-1-ethyl-4-isopropyl-1H-imidazol-2-ylmethyl]-urea

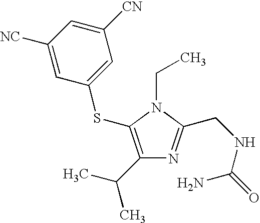

The title compound was prepared from the compound of Example 45 and trichloroacetylisocyanate, using a similar method to that of Example 4, as a white solid in 0.4% yield.

LRMS: m/z APCI-369 [M+H]⁺.

### EXAMPLE 45

### 5-(2-Aminomethyl-3-ethyl-5-isopropyl-3H-imidazol-4-ylsulfanyl)-isophthalonitrile

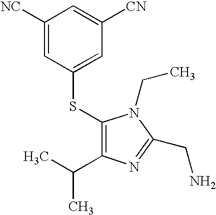

The title compound was prepared from the compound of Preparation 22, using a method similar to that of Example 25, as a white solid in 72% yield.

LRMS: m/z APCI-326 [M+H]⁺.

### EXAMPLE 46

### 5-(5-Cyclopropyl-3-ethyl-2-hydroxymethyl-3H-imidazol-4-visulfanyl)-isophthalonitrile

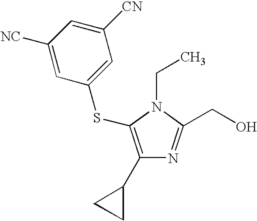

Boron trichloride-methyl sulphide complex solution (2M in dichloromethane, 1.65 mL, 3.3 mmol) was added to a solution of the compound of Preparation 37 (680 mg, 1.64 mmol) in dichloromethane (16 mL) and the mixture was stirred for 1 h at rt. The reaction was then quenched by the addition of sodium hydrogen carbonate solution (25 mL) and diluted with dichloromethane (25 mL). The layers were separated and the aqueous layer was extracted with dichloromethane (20 mL). The combined organic solutions were washed with brine (20 mL), dried over magnesium sulphate and concentrated in vacuo to give a yellow residue. The residue was purified by column chromatography on silica gel, eluting with dichloromethane:methanol, 100:0 to 98:2. The relevant fractions were evaporated under reduced pressure and the residue was re-crystallised from ethyl acetate/pentane to afford the title compound as colourless crystals in 35% yield (200 mg).

LRMS: m/z ES 325 [M+H]⁺.

### EXAMPLE 47

### 5-(5-Ethyl-2-hydroxymethyl-3-(pyridin-4-ylmethyl)-3H-imidazol-4-ylsulfanyl)-isophthalonitrile

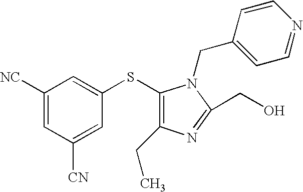

Potassium carbonate (160 mg, 1.2 mmol) was added to a solution of the compound of Preparation 23 (140 mg, 0.49 mmol) and 4-(chloromethyl)pyridine hydrochloride (89 mg, 0.54 mmol) in N,N-dimethylformamide (1.5 mL) and the suspension was stirred for 20 h at rt. The reaction mixture was then diluted with water (20 mL) and extracted into ethyl acetate (30 mL, 20 mL). The combined organic solution was washed with water (10 mL) and brine (10 mL), dried over magnesium sulphate and concentrated in vacuo. The residue was purified twice by column chromatography on silica gel, first eluting with dichloromethane:methanol: 0.88 ammonia, 100:0:0 to 95:5:0.5, then eluting with toluene:ethyl acetate:diethylamine, 1:0:0 to 0:9:1. The product was further purified by HPLC using a Chirapak AD-H reverse-phase column and a mobile phase of acetonitrile modified with 0.1% trifluoroacetic acid to afford the title compound in 13% yield (44 mg) as a colourless gum.

LRMS: m/z ESI 376 [M+H]⁺.

### Preparation 1: O-(3,5-Dicyanophenyl) dimethylthiocarbamate

3,5-Dicyanohydroxybenzene (WO02/085860) (50 g, 350 mmol) in 1-methyl-2-pyrrolidinone (200 mL) was added to a stirred suspension of sodium carbonate (48 g, 450 mmol) in 1-methyl-2-pyrrolidinone (200 mL) at 0° C. under a nitrogen atmosphere. After warming to rt, the resulting mixture was stirred at ambient temperature for 30 min. Then a solution of dimethylthiocarbamoyl chloride (56 g, 450 mmol) in 1-methyl-2-pyrrolidinone (200 mL) was added. The reaction mixture was stirred at rt for 30 min, then at 70° C. for 16 h. After this time, the reaction mixture was cooled to rt and water (200 mL) was added. The resultant white precipitate was collected by filtration and dried in a vacuum oven at 55° C. for 48 hours to give the title compound as a colourless solid (53 g).

### Preparation 2: S-(3,5-Dicyanophenyl) dimethylthiocarbamate

A sample of the compound of Preparation 1 (49.9 g, 216 mmol) was heated at 200° C. under a nitrogen atmosphere. The sample melted, started to blacken and after 1 h solidified. Heating of this solid was continued for a further 2 hours and then the reaction mixture was allowed to cool to rt and was used directly in Preparation 3 without characterisation.

### Preparation 3: 5-Mercaptoisophthalonitrile

To a stirred suspension of the compound of Preparation 2 in tetrahydrofuran (500 mL) and methanol (400 mL) at rt was added dropwise a solution of sodium hydroxide (8.6 g, 216 mmol) in methanol (100 mL). After 15 h the reaction mixture was concentrated and the residue was dissolved in water (800 mL) and was washed with dichloromethane (2×100 mL), before addition of 2M aqueous hydrochloric acid (110 mL) to the aqueous component which resulted in a cream precipitate from a yellow solution. The precipitate was collected by filtration, washed with water (100 mL) and dried by suction to give a beige powder. The crude product was recrystallised from methanol/water (approximately 1:1 by volume) to give an initial batch of the title compound as beige needles (10 g). A second batch of the title compound was obtained by dilution of the mother liquors with water to give a white powder, suitable for recrystallisation as described above.

### Preparation 4: 2-[2-(Benzyloxy)ethyl]-4-ethyl-1H-imidazole

3-Benzyloxy-1-propionaldehyde (Tetrahedron, 2000, 56, 5303-5310) (2.82 g, 17.5 mmol) was added to a stirred solution of 2,2-dichlorobutanal (Synthesis, 1975, 455-456) (2.47 g, 17.5 mmol) in acetonitrile (10 mL) at 0° C. under a nitrogen atmosphere, followed by 0.88 ammonia (20 mL). The reaction was stirred at rt for 16 h. The mixture was evaporated under reduced pressure and the residual liquid was diluted with water (10 mL), washed with dichloromethane (3×20 mL), and the combined organic fraction was dried over magnesium sulphate, filtered and evaporated under reduced pressure. The residue was purified by chromatography on silica gel using an elution gradient of dichloromethane:methanol (100:0 to 98:2 to 97:3). This afforded the title compound as a yellow oil (1.84 g).

### Preparation 5: 2-[2-(Benzyloxy)ethyl]-4-ethyl-5-iodo-1H-imidazole

To the compound of Preparation 4 (500 mg, 2.17 mmol) in dichloromethane (2.5 mL) at 0° C. was added a solution of sodium hydroxide (9.6 mg, 2.4 mmol) in water (1.5 mL). A solution of iodine (716 mg, 2.82 mmol) in a mixture of dichloromethane and methanol (5:2, 6 mL total) was added dropwise over 10 min to the vigorously stirred solution. The resulting mixture was stirred at 0° C. for 20 min after which time the reaction was quenched with the addition of a 5% aqueous sodium sulphite solution (40 mL) and the mixture was extracted with dichloromethane (3×30 mL). The combined organic fraction was washed with water (15 mL) followed by a solution of brine (20 mL), the organic fraction was dried over sodium sulphate, filtered and evaporated under reduced pressure. The residue was purified by chromatography on silica gel using an elution gradient of dichloromethane:methanol:0.88 ammonia (100:0:0 to 95:5:0.5). This afforded the title compound as a colourless gum (460 mg).

### Preparation 6: 5-[(3,5-Dicyanophenyl)dithio]isophthalonitrile

To the compound of Preparation 3 (2 g, 12.5 mmol), suspended in a solution of potassium hydroxide (580 mg, 12.5 mmol) in water (4 mL), was added, dropwise, a solution of iodine (1.59 g, 6.25 mmol) and potassium iodide (2.07 g, 12.5 mmol) in water (4 mL). The resulting mixture was stirred at rt for 16 h. The resulting solid which formed was collected by filtration and washed with water (10 mL). The wet solid was dissolved in ethyl acetate (300 mL), dried over magnesium sulphate, filtered and the solvent removed under reduced pressure. The residue was purified by chromatography on silica gel using an elution gradient of dichloromethane:ethyl acetate (100:0 to 95:5). This afforded the title compound as a white solid (800 mg).

### Preparation 7: 5-[2-(2-Benzyloxyethyl)-5-ethyl-3H-imidazol-4-ylsulfanyl]-isophthalonitrile

To the compound of Preparation 5 or 11 (450 mg, 1.26 mmol) in dry dimethylsulphoxide (2 mL) was added lithium hydride in one portion (11 mg, 1.35 mmol). The resulting mixture was stirred under a nitrogen atmosphere for 10 min, after which time the compound of Preparation 6 was added in one portion (403 mg 1.26 mmol). The resulting solution was heated at 60° C. for 3 h and then cooled to rt. The mixture was then cooled to 0° C. and water (40 mL) added. The mixture was extracted with a mixture of ethyl acetate:diethyl ether (1:1, 2×50 mL). The combined organic fraction was washed with water (15 mL), followed by a solution of brine (2×30 mL), dried over magnesium sulphate, filtered and the solvent removed under reduced pressure. The residue was purified by chromatography on silica gel using an elution gradient of dichloromethane:methanol (100:0 to 98:2). This afforded the title compound as an impure yellow oil which was subjected to a second round of silica gel chromatography using an elution gradient of n-pentane:ethyl acetate (1:1 to 1:4) to afford the title compound as a colourless gum (300 mg).

### Preparation 8: 5-[2-(2-Benzyloxyethyl)-3,5-diethyl-3H-imidazol-4-ylsulfanyl]-isophthalonitrile

To the compound of Preparation 7 or 12 (290 mg, 0.75 mmol) in N,N-dimethylformamide (1 mL) was added potassium carbonate (160 mg, 1.2 mmol) followed by ethyl iodide (125 mg, 0.8 mmol) and the resulting mixture stirred at rt for 26 hours. After this time the mixture was diluted with water (25 mL) and the mixture extracted with ethyl acetate (2×25 mL). The combined organic fraction was washed with water (5 mL) followed by a solution of brine (10 mL), dried over sodium sulphate, filtered and the solvent removed under reduced pressure. The residue was purified by chromatography on silica gel using an elution gradient of toluene:ethyl acetate (4:1 to 2:1). This afforded the title compound as the most polar isomer (eluted second from the column) as a colourless gum (120 mg).

### Preparations 9 and 10

The following compounds were prepared according to the method described above from Preparation 8 using the appropriate alkylating agent [2-(chloromethyl)-pyridine hydrochloride and iodomethane, respectively]:
Preparation 9: 5-[2-(2-Benzyloxyethyl)-5-ethyl-3-(pyridin-2-ylmethyl)-3H-imidazol-4-ylsulfanyl]-isophthalonitrile
Preparation 10: 5-[2-(2-Benzyloxyethyl)-5-ethyl-3-methyl-3H-imidazol-4-ylsulfanyl]-isophthalonitrile

### Preparation 11: 2-(2-Benzyloxyethyl)-4-ethyl-5-iodo-1H-imidazole

A solution of 3-benzyloxy-1-propionaldehyde (Tetrahedron, 2000, 56, 5303-5310) (135 g, 957 mmol) and 2,2-dichlorobutanal (Synthesis, 1975, 455-456) (154.3 g, 957 mmol) in acetonitrile (250 mL) was cooled to −5° C. under a nitrogen atmosphere and treated with 0.88 ammonia (650 mL, added in 50 mL portions). The reaction was stirred at rt for 16 h. Dichloromethane (500 mL) was added to the mixture and the layers separated. The aqueous layer was further extracted with dichloromethane (2×200 mL) and the combined organic fraction was washed with saturated brine (500 mL), dried over magnesium sulphate, filtered and evaporated under reduced pressure to give 244 g of a thick orange oil. This oil was dissolved in dichloromethane (400 mL), cooled to 0° C. and treated with a solution of sodium hydroxide (46.61 g, 1.165 mol) in water (200 mL). A slurry of iodine (295.8 g, 1.165 mol) in methanol:dichloromethane (1:1, 400 mL) was then added to the well-stirred mixture. The resulting brown-black mixture was stirred for 1 h and allowed to warm to 8° C. The mixture was diluted with dichloromethane (400 mL) and treated with 10% aqueous sodium sulphite solution (500 mL) with vigorous mixing. The layers were separated and the aqueous layer further extracted with dichloromethane (2×300 mL). The combined organic fraction was washed with 10% aqueous sodium sulphite solution (500 mL) and saturated brine (600 mL), dried over magnesium sulphate, filtered and evaporated under reduced pressure. The residue was purified by chromatography on silica gel using an elution gradient of pentane:ethyl acetate (1:1 to 0:1) to give a solid. This solid was treated with pentane (1 L), cooled and stirred; the resulting solid was collected by filtration and washed with pentane (500 mL) to afford the title compound as a white solid (117.44 g).

### Preparation 12: 5-[2-(2-Benzyloxy-ethyl)-5-ethyl-3H-imidazol-4-ylsulfanyl]-isophthalonitrile

To the compound of Preparation 5 or 11 (18.5 g, 51.97 mmol) in acetonitrile (200 mL) were added Preparation 3 (6 g, 72.5 mmol) followed by caesium carbonate (13 g, 77.85 mmol). The resulting mixture was heated at reflux for 72 h after which time the mixture was cooled to ambient temperature and water (150 mL) added. To this was added dichloromethane (250 mL) and the layers separated. The aqueous extract was washed with dichloromethane (150 mL) and the combined organic extracts were washed with a solution of brine (150 mL), dried over magnesium sulphate, filtered and the solvent removed under reduced pressure. The residue was purified by chromatography on silica gel using ethyl acetate:pentane (1:1). This afforded the title compound as an orange oil (19.34 g).

### Preparation 13: 2-Benzyloxymethyl-4-ethyl-1H-imidazole

Benzyloxyacetaldehyde (11.4 mL, 80.9 mmol) was added to a stirred solution of 2,2-dichlorobutanal (Synthesis, 1975, 455-456) (11.4 g, 80.9 mmol) in acetonitrile (40 mL) at 0° C. under a nitrogen atmosphere, followed by 0.88 ammonia (80 mL). The reaction was stirred at rt for 48 h. The mixture was then evaporated under reduced pressure and the residue was extracted with dichloromethane (300 mL, 2×100 mL). The combined organic solutions were dried over magnesium sulphate and concentrated in vacuo to give a dark brown oil. The oil was purified by chromatography on silica gel eluting with dichloromethane:methanol:0.88 ammonia, 100:0:0 to 95:5:0.5, followed by trituration with diethyl ether to afford the title compound as a pale brown solid in 53% yield (9.2 g).

### Preparation 14: 2-Benzyloxymethyl-4-trifluoromethyl-1H-imidazole

1,1,1-Trifluoro-3,3-dibromoacetone (10.4 mL, 55 mmol) was added to a solution of sodium acetate trihydrate (13.6 g, 100 mmol) in water (45 mL) and the mixture was heated at reflux for 30 min. The mixture was then cooled to rt and added to a solution of benzyloxyacetaldehyde (7.0 mL, 50 mmol) in methanol (230 mL) and 0.88 ammonia (57 mL), and the mixture was stirred at rt for 18 h. The reaction mixture was concentrated in vacuo to low volume (60 mL), diluted with water (50 mL) and triturated. The resulting precipitate was filtered off and dried in vacuo at 60° C. to afford the title compound as a pale brown solid in 92% yield (13 g).

### Preparation 15: 2-Benzyloxymethyl-4-ethyl-5-iodo-1H-imidazole

The title compound was prepared from the compound of Preparation 13 and iodine, using a method similar to that of Preparation 5, as a pale yellow gum in 65% yield.

### Preparation 16: 2-Benzyloxymethyl-5-iodo-4-trifluoromethyl-1H-imidazole

Iodine (12.0 g, 47.5 mmol), periodic acid dihydrate (10.3 g, 45 mmol) and chloroform (45 mL) were added to a solution of the compound of Preparation 14 (11.5 g, 45 mmol) in acetic acid (135 mL), and the mixture was heated at 60° C. for 4 hours. The mixture was then allowed to cool to rt and was poured onto ice-cold 10% aqueous sodium bisulphite solution (600 mL). The aqueous solution was extracted with ethyl acetate (3×400 mL) and the combined organic solution was washed with brine (400 mL), dried over magnesium sulphate and concentrated in vacuo. Purification of the residue by column chromatography on silica gel, eluting with ethyl acetate:pentane, 33:66, followed by trituration with pentane afforded the title compound as a white powder in 81% yield (14 g).

### Preparation 17: 5-(2-Benzyloxymethyl-5-ethyl-3H-imidazol-4-ylsulfanyl)-isophthalonitrile

Caesium carbonate (7.15 g, 22 mmol) was added to a stirred solution of the compounds of Preparation 3 (3.04 g, 19 mmol) and Preparation 15 (5 g, 14.6 mmol) in acetonitrile (50 mL) and the reaction mixture was heated under reflux for 90 min. The cooled mixture was then filtered and the filtrate was concentrated in vacuo to give a brown solid. The solid was combined with the filtered solid and suspended in ethyl acetate (200 mL), washed with water (200 mL), and the aqueous washing was extracted with ethyl acetate (2×100 mL). The combined organic solution was then washed with brine (2×100 mL), dried over magnesium sulphate and concentrated in vacuo to give a brown gum. The gum was purified by column chromatography on silica gel, eluting with pentane:ethyl acetate, 80:20 to 50:50 to afford the title compound as a pale yellow foam in 81% yield (4.5 g).

### Preparation 18: 5-(2-Benzyloxymethyl-5-trifluoromethyl-3H-imidazol-4-ylsulfanyl)isophthalonitrile

Caesium carbonate (4.56 g, 14 mmol) was added to a stirred solution of the compound of Preparation 3 (1.92 g, 12 mmol) in acetonitrile (80 mL) and the mixture was stirred for 5 min at rt. Preparation 16 (3.81 g, 10 mmol) was then added portionwise, followed by copper (I) iodide (570 mg, 3 mmol) and the reaction mixture was heated under reflux for 18 h. After this time, TLC analysis showed that starting material still remained and so further copper (I) iodide (570 mg, 3 mmol) was added to the mixture and heating continued for 6 h. The mixture was then cooled to rt and was concentrated in vacuo. The residue was partitioned between ethyl acetate and 10% citric acid solution and the resulting precipitate was filtered off. The layers of the filtrate were separated and the aqueous layer was extracted with ethyl acetate (3×200 mL). The combined organic solution was then washed with brine, dried over magnesium sulphate and concentrated in vacuo to give a yellow oil. Purification of the oil by column chromatography on silica gel, eluting with ethyl acetate:toluene, 20:80, afforded the title compound as a pale yellow gum in 48% yield (2 g).

### Preparation 19: 5-(2-Benzyloxymethyl-3,5-diethyl-3H-imidazol-4-ylsulfanyl)-isophthalonitrile

The title compound was prepared from the compound of Preparation 17 and ethyl iodide, using a method similar to that of preparation 8. Purification of the crude compound by column chromatography on silica gel, eluting with toluene:ethyl acetate 90:10, firstly gave 5-(2-benzyloxymethyl-1,5-diethyl-1H-imidazol-4-ylsulphanyl)-isophthalonitrile as a gum in 19% yield. Further elution then afforded the desired product, 5-(2-benzyloxymethyl-3,5-diethyl-3H-imidazol-4-ylsulphanyl)-isophthalonitrile, as a gum in 31% yield.

### Preparation 20: 5-(2-Benzyloxymethyl-3-ethyl-5-trifluoromethyl-3H-imidazol-4-ylsulfanyl)-isophthalonitrile

The title compound was prepared from the compound of Preparation 18 and ethyl iodide, using a method similar to that of preparation 8. The crude compound was further purified by trituration in diethyl ether/pentane to afford the desired product as a white solid in 70% yield.

### Preparation 21: 5-[2-(1,3-Dioxo-1,3-dihydro-isoindol-2-ylmethyl)-3,5-diethyl-3H-imidazol-4-ylsulfanyl]-isophthalonitrile

The compound of Example 6 (150 mg, 0.48 mmol) was added to a solution of triphenylphosphine (189 mg, 0.72 mmol) and phthalimide (106 mg, 0.72 mmol) in tetrahydrofuran (4 mL). The resulting solution was added dropwise to an ice-cold solution of diisopropyl azodicarboxylate (139 μL, 0.72 mmol) in tetrahydrofuran (0.5 mL) and the solution was stirred at rt for 2 h. The mixture was then diluted with ethyl acetate (50 mL) and washed with water (50 mL). The aqueous layer was extracted with further ethyl acetate (50 mL) and the combined organic solution was washed with brine, dried over magnesium sulphate and concentrated in vacuo. Purification of the residue by column chromatography on silica gel, eluting with pentane:ethyl acetate 75:25 to 50:50, afforded the title compound as a white foam in quantitative yield (235 mg).

### Preparation 22: 5-[2-(1,3-Dioxo-1,3-dihydro-isoindol-2-ylmethyl)-3-ethyl-5-isopropyl-3H-imidazol-4-ylsulfanyl]-isophthalonitrile

The title compound was prepared from the compound of Example 8 and phthalimide, using a similar method to that of Preparation 21, as a white crystalline solid in 94% yield.

### Preparations 23 to 25

The following compounds were prepared by a method similar to that described for example 6 using the appropriate starting material and boron trichloride-methyl sulphide complex solution. The reactions were monitored by TLC analysis and were stirred at rt for 3-18 h.
Preparation 23: 5-(5-Ethyl-2-hydroxymethyl-3H-imidazol-4-ylsulfanyl)-isophthalonitrile (from the compound of Preparation 17, yield 59%);
Preparation 24: 5-(2-Hydroxymethyl-5-isopropyl-3H-imidazol-4-ylsulfanyl)-isophthalonitrile (from the compound of Preparation 47, yield 60%);
Preparation 25: 5-(5-Cyclopropyl-2-hydroxymethyl-3H-imidazol-4-ylsulfanyl)-isophthalonitrile (from the compound of Preparation 60, yield 63%).

### Preparation 26: 5-Cyclopropyl-2-methyl-3H-imidazole-4-carboxylic acid ethyl ester

Acetaldehyde (2.2 mL, 40 mmol), ammonium acetate (6.2 g, 80 mmol), and triethylamine (11.2 mL, 80 mmol) were added to a solution of ethyl 3-cyclopropyl-2,2-dihydroxy-3-oxopropanoate [(3.8 g, 20 mmol) J. labelled compounds and radiopharmaceuticals, 38(5), 453, 9381-138] in tetrahydrofuran (100 mL) and the reaction mixture was stirred for 18 h at rt. The mixture was then diluted with ethyl acetate (100 mL) and quenched with brine (150 mL). The organic layer was separated and the aqueous layer was extracted with ethyl acetate (3×100 mL). The combined organic solution was washed with brine, dried over magnesium sulphate and concentrated in vacuo to give an orange residue. Trituration of the residue with diethyl ether afforded the title compound as a pale orange solid in 46% yield (1.8 g).

### Preparation 27: 2-Benzyloxymethyl-5-cyclopropyl-3H-imidazole-4-carboxylic acid ethyl ester

Benzyloxyaldehyde (192 μL, 1.36 mmol), ammonium acetate (358 mg, 4.64 mmol), and triethylamine (650 μL, 4.64 mmol) were added to a solution of ethyl 3-cyclopropyl-2,2-dihydroxy-3-oxopropanoate [(213 mg, 1.13 mmol) J. labelled compounds and radiopharmaceuticals, 38(5), 453-470; 1996] in tetrahydrofuran (5 mL) and the reaction mixture was heated under reflux for 1 h. The mixture was then diluted with dichloromethane (50 mL) and quenched with brine (50 mL). The organic layer was separated and the aqueous layer was extracted with dichloromethane (2×25 mL). The combined organic solution was washed with brine (25 mL), dried over magnesium sulphate and concentrated in vacuo. The residue was purified by column chromatography on silica gel, eluting with pentane:ethyl acetate, 67:33 to 50:50 to afford the title compound as a solid in 93% yield (290 mg).

### Preparation 28: 2-(2-Benzyloxy-ethyl)-5-cyclopropyl-3H-imidazole-4-carboxylic acid ethyl ester

The title compound was prepared from ethyl 3-cyclopropyl-2,2-dihydroxy-3-oxopropanoate (J. labelled compounds and radiopharmaceuticals, 38(5), 453-470; 1996) and 4-(phenylmethoxy)propanal (Tetrahedron, 56, 5303-5310; 2000), using a similar method to that of Preparation 26, as a yellow oil in 70% yield.

### Preparation 29: 2-Benzyloxymethyl-5-cyclopropyl-3-ethyl-3H-imidazole-4-carboxylic acid ethyl ester

To the compound of Preparation 27 (100 mg, 0.33 mmol) in N,N-dimethylformamide (1 mL) was added potassium carbonate (69 mg, 0.5 mmol) followed by ethyl iodide (30 μL, 0.37 mmol) and the resulting mixture stirred at rt for 25 h. The solvent was then evaporated under reduced pressure and the residue was partitioned between ethyl acetate (25 mL) and brine (25 mL). The organic layer was washed with brine (25 mL) dried over magnesium sulphate, filtered and concentrated in vacuo to afford the title compound as a yellow oil in 90% yield (100 mg).

### Preparation 30: 2-(2-Benzyloxy-ethyl)-5-cyclopropyl-3-ethyl-3H-imidazole-4-carboxylic acid ethyl ester

Ethyl iodide (546 μL, 6.82 mmol) was added to a suspension of the compound of Preparation 28 (1.95 g, 6.2 mmol) and potassium carbonate (1.28 g, 9.3 mmol) in N,N-dimethylformamide (20 mL) and the mixture was stirred for 18 h at rt. Further ethyl iodide (50 μL, 0.62 mmol) was added and the mixture was stirred for 3 h at rt. The solvent was then evaporated under reduced pressure and the residue was partitioned between ethyl acetate (150 mL) and brine (150 mL). The layers were separated and the aqueous layer was extracted with ethyl acetate (2×50 mL). The combined organic solution was washed with brine, dried over magnesium sulphate and concentrated in vacuo to afford the title compound as a yellow oil in 85% yield (1.77 g).

### Preparation 31: 2-Benzyloxymethyl-4-cyclopropyl-1-ethyl-5-iodo-1H-imidazole

1M Sodium hydroxide solution (44 mL, 44 mmol) was added to a solution of the compound of Preparation 29 (1.45 g, 4.4 mmol) in methanol (120 mL) and the mixture was heated under reflux for 3 h. The reaction mixture was then cooled to rt and the solvent was evaporated under reduced pressure. The residue was diluted with dichloromethane (120 mL) and brine (100 mL), and the mixture was adjusted to pH 12 with 2M hydrochloric acid. A solution of iodine (1.46 g, 5.74 mmol) in dichloromethane (35 mL) was added dropwise to the vigorously stirred biphasic system and the pH was maintained at 12 by the periodic addition of 1 M sodium hydroxide solution. After stirring for 18 h, 10% sodium thiosulphate solution was added and the resulting mixture was neutralised by the addition of 2M hydrochloric acid. The organic layer was separated and the aqueous layer was extracted with dichloromethane. The combined organic solution was then washed with brine, dried over magnesium sulphate and concentrated in vacuo to give a yellow oil. The oil was purified by column chromatography on silica gel, eluting with pentane:ethyl acetate 83:17, to afford the title compound as a colourless glass in 55% yield (905 mg).

### Preparations 32 to 34

The following compounds were prepared by a method similar to that described for preparation 31, using the appropriate starting material and iodine.
Preparation 32: 4-Cyclopropyl-5-iodo-2-methyl-1H-imidazole (from the compound of Preparation 26, yield 58%);
Preparation 33: 2-(2-Benzyloxyethyl)-4-cyclopropyl-5-iodo-1H-imidazole (from the compound of Preparation 28, yield 43%);
Preparation 34: 2-(2-Benzyloxyethyl)-4-cyclopropyl-1-ethyl-5-iodo-1H-imidazole (from the compound of Preparation 29, yield 40%, purified by column chromatography on silica gel, eluting with pentane:ethyl acetate, 83:17 to 67:33).

### Preparations 35 and 36

The following compounds were prepared by a method similar to that described for Preparation 17, using the appropriate iodo-imidazole and mercaptoisophthalonitrile (preparation 3).
Preparation 35: 5-(5-Cyclopropyl-2-methyl-3H-imidazol-4-ylsulfanyl)-isophthalonitrile (from preparation 32, yield 67%);
Preparation 36: 5-[2-(2-Benzyloxyethyl)-5-cyclopropyl-3H-imidazol-4-ylsulfanyl]-isophthalonitrile (from preparation 33, yield 93%).

### Preparation 37: 5-(2-Benzyloxymethyl-5-cyclopropyl-3-ethyl-3H-imidazol-4-ylsulfanyl)-isophthalonitrile

Caesium carbonate (842 mg, 2.59 mmol) was added to a solution of the compound of Preparation 3 (378 mg, 2.35 mmol) in acetonitrile (15 mL) and the mixture was stirred for 10 min. The compound of Preparation 31 (900 mg, 2.35 mmol) and copper (I) iodide (135 mg, 0.7 mmol) were then added and the mixture was heated under reflux for 18 h. The reaction mixture was cooled to rt, filtered and concentrated in vacuo, and the residue was partitioned between ethyl acetate (70 mL) and brine (70 mL). The layers were then separated and the aqueous layer was extracted with ethyl acetate (50 mL). The combined organic solution was filtered, dried over magnesium sulphate and concentrated in vacuo to give an orange oil. The oil was purified by column chromatography on silica gel, eluting with ethyl acetate:pentane, 25:75, to afford the title compound as a pale yellow oil in 75% yield (705 mg).

### Preparation 38: 5-[2-(2-Benzyloxyethyl)-5-cyclopropyl-3-ethyl-3H-imidazol-4-ylsulfanyl]-isophthalonitrile

The title compound was prepared from the compounds of Preparation 34 and Preparation 3, using a method similar to that of Preparation 18. The crude compound was purified by column chromatography on silica gel, eluting with pentane:ethyl acetate, 75:25 to 50:50, to afford the desired product in 75% yield.

### Preparation 39: 5-[5-Cyclopropyl-2-(2-hydroxy-ethyl)-3H-imidazol-4-ylsulfanyl]-isophthalonitrile

The title compound was prepared from the compound of Preparation 36, using a similar method to that of example 5, as a solid in 95% yield.

### Preparation 40: 5-[5-Ethyl-2-(2-hydroxy-ethyl)-3H-imidazol-4-ylsulfanyl]-isophthalonitrile

The title compound was prepared from the compound of Preparation 7, using a similar method to that of example 6, as a solid in 74% yield.

### Preparation 41: 2,2-Dichloro-3-methyl-butyraldehyde

Isovaleraldehyde (13.4 mL, 0.13 mol) was added dropwise to sulfuryl chloride (80 mL, μmol) at rt. The mixture was warmed to 30° C. and stirred for 15 min before re-cooling to rt. Further isovaleraldehyde (13.4 mL, 0.13 mol) was then added dropwise and the reaction mixture was left to stand for 15 min before heating under gentle reflux for 18 h. The reaction mixture was then distilled and the fraction containing the title compound (12.43 g, 32% yield) was collected in the range of 140-147° C.

### Preparation 42: 2-(2-Benzyloxy-ethyl)-4-isopropyl-1H-imidazole

4-(Benzyloxy)propanal [(13.37 g, 0.08 mol), Tetrahedron, 56, 5303-5310; 2000)] was added to an ice-cold solution of preparation 41 (12.4 g 0.08 mol) in acetonitrile (40 mL). 0.88 Ammonia (80 mL) was then added portionwise and the mixture was stirred at rt for 48 h. The solvent was evaporated under reduced pressure and the residue was partitioned between dichloromethane (400 mL) and water (150 mL). The aqueous phase was separated and extracted with dichloromethane (250 mL) and the combined organic solution was washed with brine (300 mL), dried over magnesium sulphate and concentrated in vacuo. The residue was purified by column chromatography on silica gel, eluting with dichloromethane:methanol, 98:2, to afford the title compound as a viscous orange oil in 38% yield (7.64 g).

### Preparation 43: 2-Benzyloxymethyl-4-isopropyl-1H-imidazole

Benzyloxyacetaldehyde (33.54 g, 0.22 mol) was added to an ice-cold solution of the compound of Preparation 41 (34 g 0.22 mol) in acetonitrile (150 mL). 0.88 Ammonia (230 mL) was then added portionwise and the mixture was stirred rt for 48 h. The solvent was evaporated under reduced pressure and the residue was partitioned between dichloromethane (600 mL) and water (200 mL). The aqueous layer was separated and extracted with dichloromethane (250 mL) and the combined organic solution was washed with brine (400 mL), dried over magnesium sulphate and concentrated in vacuo. The residue was purified by column chromatography on silica gel, eluting with dichloromethane:methanol, 98:2 to 95:5, to afford the title compound as a viscous orange oil in 36% yield (18.56 g).

### Preparation 44: 2-(2-Benzyloxy-ethyl)-5-iodo-4-isopropyl-1H-imidazole

Sodium hydroxide solution (30 mL, 36.2 mmol) was added portionwise to an ice-cold solution of the compound of Preparation 42 (7.64 31.1 mmol) in dichloromethane (100 mL). A slurry of iodine (8.74 g, 34.4 mmol) in a mixture of dichloromethane (25 mL) and methanol (25 mL) was added and the mixture was stirred at 0° C. for 20 min. The mixture was then partitioned between 10% sodium thiosulphate solution (150 mL) and dichloromethane (200 mL). The aqueous phase was separated and extracted with dichloromethane (150 mL) and the combined organic solution was washed with brine (350 mL), dried over magnesium sulphate and concentrated in vacuo. Purification of the residue by column chromatography on silica gel, eluting with dichloromethane:methanol, 98:2, followed by trituration in pentane afforded the title compound as a solid in 56% yield (6.45 g).

### Preparation 45: 2-Benzyloxymethyl-5-iodo-4-isopropyl-1H-imidazole

Sodium hydroxide solution (20 mL, 34.4 mmol) was added portionwise to an ice-cold solution of the compound of Preparation 43 (8.36 g, 36.2 mmol) in dichloromethane (100 mL). A slurry of iodine (10.13 g, 39.9 mmol) in a mixture of dichloromethane (20 mL) and methanol (30 mL) was added and the mixture was stirred at 0° C. for 15 min and at rt for 20 min. The mixture was then partitioned between 10% sodium thiosulphate solution (150 mL) and dichloromethane (200 mL). The aqueous layer was separated and extracted with dichloromethane (200 mL) and the combined organic solution was washed with brine (350 mL), dried over magnesium sulphate and concentrated in vacuo. The oily residue was then triturated in pentane to afford the title compound as a pale yellow solid in 76% yield (9.88 g).

### Preparation 46: 2-(5-Iodo-4-isopropyl-1H-imidazol-2-yl)-ethanol

A mixture of the compound of Preparation 44 (700 mg, 1.8 mmol) and concentrated hydrochloric acid (3 mL) in ethanol (1.2 mL) were heated under reflux for 18 h. The solvent was then evaporated under reduced pressure and the residue was partitioned between water (20 mL) and pentane (20 mL). The layers were separated and the aqueous solution was basified with sodium hydrogen carbonate and extracted with ethyl acetate (2×50 mL). The combined organic solution was washed with brine, dried over sodium sulphate and concentrated in vacuo to afford the title compound as a yellow solid in 93% yield (492 mg).

### Preparation 47: 5-(2-Benzyloxymethyl-5-isopropyl-3H-imidazol-4-ylsulfanyl)-isophthalonitrile

The compound of Preparation 3 (3.15 g, 19.6 mmol) was added to a stirred suspension of caesium carbonate (7.15 g, 22 mmol) and the compound of Preparation 45 (5 g, 14 mmol) in acetonitrile (100 mL) and the reaction mixture was heated under reflux for 2 h. The mixture was then cooled to rt and filtered, washing through with acetonitrile. The filtrate was concentrated in vacuo and the residue was partitioned between ethyl acetate (150 mL) and water (150 mL). The organic layer was separated, washed with brine, dried over magnesium sulphate and concentrated in vacuo to afford the title compound as a brown foam in 98% yield (5.3 g).

### Preparation 48: 5-[2-(2-Hydroxy-ethyl)-5-isopropyl-3H-imidazol-4-ylsulfanyl]-isophthalonitrile

The title compound was prepared from the compounds of Preparation 46 and Preparation 3, using a method similar to that of Preparation 47, as a brown foam in 75% yield.

### Preparation 49: 5-(2-Benzyloxymethyl-3-ethyl-5-isopropyl-3H-imidazol-4-ylsulfanyl)-isophthalonitrile

Ethyl iodide (335 μL, 4.2 mmol) was added to a suspension of preparation 47 (1.47 g, 3.8 mmol) and potassium carbonate (7.87 mg, 5.7 mmol) in N,N-dimethylformamide (20 mL) and the mixture was stirred for 18 h at rt. The solvent was then evaporated under reduced pressure and the residue was partitioned between ethyl acetate (50 mL) and water (80 mL). The layers were separated and the aqueous layer was extracted with ethyl acetate (50 mL). The combined organic solution was washed with brine, dried over magnesium sulphate and concentrated in vacuo to give a brown oil. The oil was purified by column chromatography on silica gel, eluting with pentane:ethyl acetate, 75:25 to 67:33, to afford the title compound (eluted second from column) as a yellow oil in 50% yield (730 mg).

### Preparation 50: 5-(2-Benzyloxymethyl-5-isopropyl-3-methyl-3H-imidazol-4-ylsulfanyl)-isophthalonitrile

The title compound was prepared from the compound of Preparation 47 and methyl iodide, using a similar method to that of Preparation 49, as a white solid in 35% yield.

### Preparation 51: 2-Methyl-5-trifluoromethyl-1H-imidazole

A mixture of sodium acetate trihydrate (2.7 g, 20 mmol) and 1-dibromo-3,3,3-trifluoroacetone (2.7 g, 10 mmol) in water (18 mL) was heated under reflux for 30 min. The mixture was then cooled to rt and was slowly added to a solution of acetaldehyde (0.5 mL, 9 mmol) and 0.88 ammonia (11 mL) in methanol (45 mL). The mixture was stirred for 18 h at rt and was then concentrated under reduced pressure. The aqueous residue was diluted with water (10 mL) and was left to stand for 3 h. The resulting crystals were filtered off and dried to afford some title compound (0.85 g). The filtrate was then extracted with dichloromethane (3×20 mL) and the combined organic solution was dried over magnesium sulphate and concentrated in vacuo to afford further title compound (0.2 g, total yield 70%).

### Preparation 52: 2-(2-Benzyloxy-ethyl)-5-trifluoromethyl-1H-imidazole

A mixture of sodium acetate trihydrate (2.7 g, 20 mmol) and 1-dibromo-3,3,3-trifluoroacetone (2.7 g, 10 mmol) in water (18 mL) was heated under reflux for 30 min. The mixture was then cooled to rt and was slowly added to a solution of 4-(benzyloxy)propanal (Tetrahedron, 56, 5303-5310; 2000) (1.48 g, 9 mmol) and 0.88 ammonia (11 mL) in methanol (45 mL). The mixture was stirred at rt for 18 h and was then evaporated under reduced pressure. The aqueous residue was extracted with ethyl acetate (3×50 mL) and the combined organic solution was dried over magnesium sulphate and concentrated in vacuo to give an oil. The oil was then triturated in water with a trace of methanol to afford the title compound as a crystalline solid in 88% yield (2.4 g).

### Preparations 53 and 54

The following compounds were prepared by a method similar to that described for Preparation 16 using the appropriate imidazole, periodic acid and iodine.
Preparation 53: 4-Iodo-2-methyl-5-trifluoromethyl-1H-imidazole (from the compound of Preparation 51, yield 81%);
Preparation 54: 2-(2-Benzyloxyethyl)-4-iodo-5-trifluoromethyl-1H-imidazole (from the compound of Preparation 52, yield 55%).

### Preparations 55 and 56

The following compounds were prepared by a method similar to that described for Preparation 18 using the appropriate iodo-imidazole and the compound of Preparation 3.
Preparation 55: 5-(2-Methyl-5-trifluoromethyl-1H-imidazol-4-ylsulfanyl)-isophthalonitrile (from the compound of Preparation 53, yield 77%);
Preparation 56: 5-[2-(2-Benzyloxyethyl)-5-trifluoromethyl-1H-imidazol-4-ylsulfanyl]-isophthalonitrile (from the compound of Preparation 54, yield 51%);

### Preparation 57 and 58

The following compounds were prepared by a method similar to that described for Preparation 8 using the appropriate starting material and alkyl halide.
Preparation 57: 5-[2-(2-Benzyloxyethyl)-3-ethyl-5-trifluoromethyl-3H-imidazol-4-ylsulfanyl]-isophthalonitrile (from the compound of Preparation 56, yield 89%).
Preparation 58: 5-[2-(2-Benzyloxyethyl)-3-methyl-5-trifluoromethyl-3H-imidazol-4-ylsulfanyl)-isophthalonitrile (from the compound of Preparation 56, yield 98%).

### Preparation 59: 2-Benzyloxymethyl-5-iodo-4-cyclopropyl-1H-imidazole

The title compound was prepared from the compound of Preparation 27, using a similar method to that of Preparation 32, as a pale yellow oil in 70% yield.

### Preparation 60: 5-(2-Benzyloxymethyl-5-cyclopropyl-3H-imidazol-4-ylsulfanyl)-isophthalonitrile

The title compound was prepared from the compounds of Preparation 3 and Preparation 59, using a similar method to that of Preparation 17, as a white foam in 90% yield.

### Biological Data

The activity of the compounds of the invention as reverse transcriptase inhibitors may be measured using the following assay.

### Inhibition of HIV-1 Reverse Transcriptase Enzyme

The reverse transcriptase activity of the compounds of the invention may be assayed as follows. Using the purified recombinant HIV-1 reverse transcriptase (RT, EC, 2.7.7.49) obtained by expression in Escherichia Coli, a 384-well plate assay system was established for assaying a large number of samples using the [3H]-Flashplate enzyme assay system (NEN-SMP 410A) following the manufacturer's recommendations. The compounds were dissolved in 100% DMSO and diluted with the appropriate buffer to a 5% final DMSO concentration. The inhibitory activity was expressed in percent inhibition relative to the DMSO control. The concentration at which the compound inhibited the reverse transcriptase by 50% was expressed as the IC₅₀ of the compound.

All the Examples of the invention have IC₅₀ values, according to the above method, of less than 1.5 μM, as illustrated in the table below:

| | Example | | | | | | |
|---|---|---|---|---|---|---|---|
| | 1 | 4 | 9 | 14 | 23 | 38 | 40 |
| IC₅₀ (nM) | 38 | 926 | 1160 | 353 | 147 | 1070 | 135 |

1-15. (canceled)

16. A compound of formula (I), 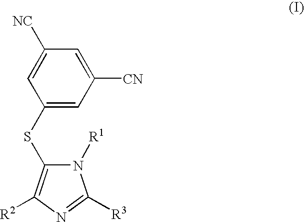
or a pharmaceutically acceptable salt or solvate, wherein:
R¹ is C₁₋₄ alkyl or C₃₋₆ cycloalkyl, wherein said alkyl is optionally substituted by pyridyl or pyridyl N-oxide;
R² is C₁₋₄ alkyl, C₃₋₆ cycloalkyl, or trifluoromethyl;
R³ is —(CH₂)ₘOR⁴, —(CH₂)ₘOC(O)NH₂, —(CH₂)ₘNH₂, or —(CH₂)ₘNHC(O)NH₂;
R⁴ is H or C₁₋₄ alkyl; and
m is 1, 2, 3 or 4.

17. A compound according to claim 16, or a pharmaceutically acceptable salt or solvate thereof, wherein R¹ is methyl, ethyl, i-propyl, cyclopropyl, or pyridylmethyl.

18. A compound according to claim 16, or a pharmaceutically acceptable salt or solvate thereof, wherein R² is methyl, ethyl, n-propyl, i-propyl, cyclopropyl, or trifluoromethyl.

19. A compound according to claim 16, or a pharmaceutically acceptable salt or solvate thereof, wherein R³ is —(CH₂)ₘOR⁴ or —(CH₂)ₘOC(O)NH₂.

20. A compound according to claim 16, or a pharmaceutically acceptable salt or solvate thereof, wherein R⁴ is H.

21. A compound according to claim 16, or a pharmaceutically acceptable salt or solvate thereof, selected from the group consisting of:
5-[3,5-Diethyl-2-(2-hydroxyethyl)-3H-imidazol-4-ylsulfanyl]-isophthalonitrile;
5-[5-Cyclopropyl-3-ethyl-2-(2-hydroxyethyl)-3H-imidazol-4-ylsulfanyl]-isophthalonitrile;
5-[3-Ethyl-2-hydroxymethyl-5-isopropyl-3H-imidazol-4-ylsulfanyl]-isophthalonitrile;
5-[3-Ethyl-2-(2-hydroxyethyl)-5-trifluoromethyl-3H-imidazol-4-ylsulfanyl]-isophthalonitrile;
Carbamic acid 4-Cyclopropyl-5-(3,5-dicyano-phenylsulfanyl)-1-ethyl-1H-imidazol-2-ylmethyl ester;
Carbamic acid 5-(3,5-Dicyano-phenylsulfanyl)-1-ethyl-4-isopropyl-1H-imidazol-2-ylmethyl ester;
Carbamic acid 5-(3,5-dicyano-phenylsulfanyl)-1,4-diethyl-1H-imidazol-2-ylmethyl ester;
Carbamic acid 5-(3,5-dicyano-phenylsulfanyl)-1-ethyl-4-(trifluoromethyl)-1H-imidazol-2-ylmethyl ester;
5-[2-Hydroxymethyl-5-isopropyl-3-(pyridin-4-ylmethyl)-3H-imidazol-4-ylsulfanyl]-isophthalonitrile;
5-[2-(2-Hydroxyethyl)-5-isopropyl-3-methyl-3H-imidazol-4-ylsulfanyl]-isophthalonitrile; and
5-[3-Ethyl-2-(2-hydroxyethyl)-5-isopropyl-3H-imidazol-4-ylsulfanyl]-isophthalonitrile;

22. A pharmaceutical composition, comprising a compound according to claim 16, or a pharmaceutically acceptable salt or solvate thereof, and one or more pharmaceutically acceptable excipients, diluents or carriers.

23. A pharmaceutical composition according to claim 22, further comprising one or more additional therapeutic agents selected from HIV protease inhibitors, non-nucleoside reverse transcriptase inhibitors, nucleoside reverse transcriptase inhibitors, CCR⁵ antagonists, CXCR⁴ antagonists, integrase inhibitors, fusion inhibitors, and RNaseH inhibitors.

24. A method of treating a mammal infected with HIV, comprising administering to said mammal an effective amount of a compound according to claim 16, or a pharmaceutically acceptable salt or solvate thereof.

25. A method for preparing a compound of formula (I), 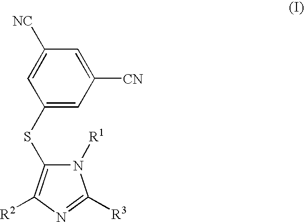
or a pharmaceutically acceptable salt or solvate, wherein:
R¹ is C₁₋₄ alkyl or C₃₋₆ cycloalkyl, wherein said alkyl is optionally substituted by pyridyl or pyridyl N-oxide;
R² is C₁₋₄ alkyl, C₃₋₆ cycloalkyl, or trifluoromethyl;
R³ is —(CH₂)ₘOR⁴—(CH₂)ₘOC(O)NH₂, —(CH₂)ₘNH₂, or —(CH₂)ₘNHC(O)NH₂;
R⁴ is H or C₁₋₄ alkyl; and
m is 1, 2, 3 or 4;
said method comprising alkylating a compound of formula (II), 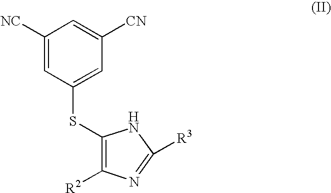
wherein R² and R³ are as hereinbefore defined, with a compound of formula with R¹X, wherein R¹ is as hereinbefore defined, and X is selected from halo, —OH, and a suitable leaving group.

26. A method for preparing a compound of formula (I), 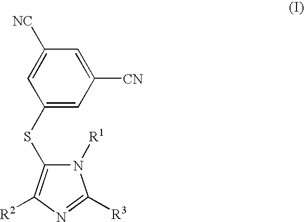
or a pharmaceutically acceptable salt or solvate, wherein:
R¹ is C₁₋₄ alkyl or C₃₋₆ cycloalkyl, wherein said alkyl is optionally substituted by pyridyl or pyridyl N-oxide;
R² is C₁₋₄ alkyl, C₃₋₆ cycloalkyl, or trifluoromethyl;
R³ is —(CH₂)ₘOR⁴—(CH₂)ₘOC(O)NH₂, —(CH₂)ₘNH₂, or —(CH₂)ₘNHC(O)NH₂;
R⁴ is H or C₁₋₄ alkyl; and
m is 1, 2, 3 or 4;
said method comprising reacting a compound of formula (XIII), 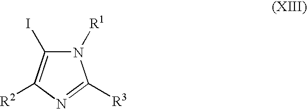
wherein R¹, R², and R³ are as hereinbefore defined, with a compound of formula (IV) or (V), [chemical structure]
